(19) United States
(12) Patent Application Publication
(10) Pub. No.: US 2024/0000758 A1
(43) Pub. Date: Jan. 4, 2024

(54) ELEVATION OF MITOCHONDRIAL BIOGENESIS AND FUNCTION BY INHIBITION OF PROSTAGLANDIN DEGRADING ENZYME 15-PGDH

(71) Applicant: The Board of Trustees of the Leland Stanford Junior University, Stanford, CA (US)

(72) Inventors: Helen M. BLAU, Stanford, CA (US); Adelaida Rosa PALLA, Stanford, CA (US); Meenakshi RAVICHANDRAN, Stanford, CA (US)

(73) Assignee: The Board of Trustees of the Leland Stanford Junior University, Stanford, CA (US)

(21) Appl. No.: 18/249,948

(22) PCT Filed: Oct. 22, 2021
(86) PCT No.: PCT/US2021/071991

§ 371 Date: Apr. 20, 2023

Related U.S. Application Data
(60) Provisional application No. 63/105,182, filed on Oct. 23, 2020.

(51) Int. Cl. A61K 31/4365; A61P 3/00

(52) U.S. Cl. CPC A61K 31/4365; A61P 3/00

(57) ABSTRACT

The present disclosure provides methods of increasing mitochondrial biogenesis, function, or both in a tissue of a subject by administering to the subject an amount of a 15-PGDH inhibitor effective to inhibit 15-PGDH activity and/or reduce 15-PGDH levels in the subject. The methods described herein are useful for treating diseases, disorders, or conditions associated with dysfunctional mitochondria and/or increased oxidative stress including mitochondrial diseases.

### CROSS-REFERENCE

This application claims priority to U.S. Provisional Patent Application No. 63/105,182, filed Oct. 23, 2020, the disclosure of which is herein incorporated by reference in its entirety.

### STATEMENT AS TO RIGHTS TO INVENTIONS MADE UNDER FEDERALLY SPONSORED RESEARCH AND DEVELOPMENT

This invention was made with Government support under contracts AG020961 and AG069858 awarded by the National Institutes of Health. The Government has certain rights in the invention.

### BACKGROUND

Mitochondrial diseases are a group of disorders caused by dysfunctional mitochondria, the organelles that generate energy for the cell. Mitochondria are found in every cell of the human body except red blood cells, and convert the energy of food molecules into the ATP that powers most cell functions.

Oxidative stress is characterized by an overproduction of reactive oxygen species (ROS) by dysfunctional mitochondria, which in turn leads to more mitochondrial damage by inducing mitochondrial DNA mutations, damaging the mitochondrial respiratory chain, changing mitochondrial membrane permeability and structure, disrupting Ca²⁺ homeostasis, and undermining mitochondrial defense systems. Oxidative stress and mitochondrial damage have been implicated in the pathogenesis of many diseases, including metabolic diseases, neurodegenerative diseases, and cancer.

However, treatment options for mitochondrial dysfunction and oxidative stress are currently limited. Thus, there remains a need in the art for effective strategies to reduce mitochondrial dysfunction and oxidative stress for the treatment of mitochondrial diseases. The present disclosure satisfies this need and provides other advantages as well.

### BRIEF SUMMARY

There remains a need in the art for effective treatments for mitochondrial diseases, disorders, and conditions. The present disclosure satisfies this need and provides other advantages as well.

The present disclosure provides methods of increasing mitochondrial biogenesis, mitochondrial function, or both in a tissue of a subject by administering to the subject an amount of a 15-hydroxyprostaglandin dehydrogenase (15-PGDH) inhibitor effective to inhibit 15-PGDH activity and/or reduce 15-PGDH levels in the subject, thereby increasing mitochondrial biogenesis, mitochondrial function, or both in the tissue of the subject. In some embodiments, the method comprises increasing mitochondrial biogenesis. In some embodiments, the method comprises increasing mitochondrial function.

In any of the embodiments, the administering comprises modulating one or more energy biomarkers, normalizing one or more energy biomarkers, or enhancing one or more energy biomarkers. In some embodiments, the one or more energy biomarkers are selected from the group consisting of: whole blood, plasma, cerebrospinal fluid, and/or cerebral ventricular fluid lactic acid (lactate) levels; whole blood, plasma, cerebrospinal fluid, and/or cerebral ventricular fluid pyruvic acid (pyruvate) levels; whole blood, plasma, cerebrospinal fluid, and/or cerebral ventricular fluid lactate/pyruvate ratios; phosphocreatine levels; NADH (NADH+H+) levels; NADPH (NADPH+H+) levels; NAD levels; NADP levels; ATP levels; reduced coenzyme Q (CoQred) levels; oxidized coenzyme Q (CoQox) levels; total coenzyme Q (CoQlot) levels; oxidized cytochrome C levels; reduced cytochrome C levels; oxidized cytochrome C/reduced cytochrome C ratio; acetoacetate levels; beta-hydroxy butyrate levels; acetoacetate/beta-hydroxy butyrate ratio; 8-hydroxy-2′-deoxyguanosine (8-OHdG) levels; levels of reactive oxygen species; levels of oxygen consumption (VO2); levels of carbon dioxide output (VCO2); respiratory quotient (VCO2/VO2); exercise tolerance; and anaerobic threshold. The methods described herein are useful for treating diseases and/or disorders associated with dysfunctional mitochondria and/or increased oxidative stress including mitochondrial diseases and/or disorders.

In some embodiments, the subject has a mitochondrial disease, disorder, or condition. In some embodiments, the mitochondrial disease, disorder, or condition is caused by mitochondrial dysfunction and/or oxidative stress. In some embodiments, the administering reduces mitochondrial dysfunction and/or oxidative stress in the tissue of the subject. In an embodiment, the mitochondrial disease, disorder, or condition is selected from the group consisting of a metabolic disease, a neurodegenerative disease, cancer, a motor neuron disease, a cardiovascular disease, a neurobehavioral or psychiatric disease, an autoimmune disease, a chronic kidney disease, myoclonic epilepsy with ragged red fibers (MERRF), mitochondrial myopathy, dominant optic atrophy (DOA), Leigh syndrome, Kearns-Sayre Syndrome (KSS), Friedrich's ataxia (FRDA), cardiomyopathy, renal tubular acidosis, Parkinson's disease, Alzheimer's disease, amyotrophic lateral sclerosis (ALS), Huntington's disease, developmental pervasive disorders, hearing loss, mitochondrial encephalopathy lactic acidosis and stroke-like episodes (MELAS), chronic obstructive pulmonary disease (COPD), ventilator induced diaphragmatic dysfunction (VIDD), myalgic encephalomyclitis/chronic fatigue syndrome. Leber hereditary optic neuropathy (LHON), chemotherapy-induced peripheral neuropathy, and a combination thereof.

In some embodiments, the administering increases a level of PGE2 in the tissue of the subject. In some embodiments, a level of PGE2 in the tissue is increased relative to the tissue prior to the administering of the 15-PGDH inhibitor. In some embodiments, a level of PGE2 in the tissue is increased to a level substantially similar to a level present in the same tissue of a subject not having the mitochondrial disease, disorder, or condition.

In any of the above embodiments, a level of PGE2 in the tissue is increased to a level substantially similar to a level present in the same tissue of a subject not having the mitochondrial disease, disorder, or condition. In some embodiments, a level of PGE2 in the tissue is increased to a level within 50% of a level present in the same tissue of a subject not having the mitochondrial disease, disorder, or condition.

In some embodiments, the tissue is selected from the group consisting of skeletal muscle, epidermal tissue, epithelial tissue, vascular tissue, cardiac muscle, brain, bone, cartilage, sensory organs, kidney, thyroid, lung, smooth muscle, brown fat, spleen, liver, heart, small intestine, colon, skin, ovaries and other reproductive tissues, hair, dental tissue, blood, cochlea, and a combination thereof.

In some embodiments, mitochondrial biogenesis, function, or both in the tissue is increased relative to the tissue prior to the administering of the 15-PGDH inhibitor. In certain embodiments, the methods described herein result in an increased level of peroxisome proliferator-activated receptor gamma coactivator 1-alpha (Pgc1α) in the tissue, increased mitochondrial DNA relative to nuclear DNA in the tissue, increased number of mitochondria in the tissue, improved mitochondrial morphology in the tissue, increased mitochondrial complexes, enzymes involved in ATP generation, electron transport, and/or respiration in mitochondria present in the tissue, increased activity of complexes I, II, III, IV, and/or V of the electron transport chain in mitochondria present in the tissue, increased mitochondrial membrane potential in the tissue, increased mitophagy or autophagy leading to mitochondria turnover in the tissue, or any combination thereof.

In some embodiments, the method described herein results in a decreased level of a PGE2 metabolite in the tissue relative to the tissue prior to the administering of the 15-PGDH inhibitor. In some embodiments, the method described herein results in a level of a PGE2 metabolite in the tissue that is substantially similar to a level present in the same tissue of a subject not having the mitochondrial disease, disorder, or condition. In some embodiments, the PGE2 metabolite is selected from the group consisting of 15-keto PGE2 and 13,14-dihydro-15-keto PGE2.

In some embodiments, the 15-PGDH inhibitor is selected from the group consisting of a small molecule compound, a blocking antibody, a nanobody, and a peptide. In some embodiments, the 15-PGDH inhibitor is SW033291. In some embodiments, the 15-PGDH inhibitor is selected from the group consisting of an antisense oligonucleotide, microRNA, siRNA, and shRNA. In some embodiments, the 15-PGDH inhibitor reduces or blocks 15-PGDH expression. In some embodiments, the 15-PGDH inhibitor reduces or blocks enzymatic activity of 15-PGDH.

In some embodiments, the subject is a human. In some embodiments, the subject is less than 30 years of age. In some embodiments, the subject is at least 30 years of age.

The present disclosure also provides methods of improving a metabolic function in a tissue of a subject having an abnormal metabolism, the method comprising administering to the subject an amount of a 15-PGDH inhibitor effective to inhibit 15-PGDH activity and/or reduce 15-PGDH levels in the subject, thereby improving the metabolic function in the tissue of the subject having the abnormal metabolism.

In some embodiments, the abnormal metabolism comprises a metabolic disorder or an obesity-related disorder. In some embodiments, the metabolic disorder is Metabolic syndrome (MS). In some embodiments, the metabolic disorder or obesity-related disorder comprises one or more of: insulin resistant obesity; diabetes; hyperphagia; endocrine abnormalities; triglyceride storage disease; Bardet-Biedl syndrome; Lawrence-Moon syndrome; Prader-Labhard-Willi syndrome; anorexia; and cachexia. In any embodiment, the disorder may result in or cause obesity. In some embodiments, the disorder that results in or causes obesity is selected from the group comprising: overeating and bulimia, polycystic ovarian disease, craniopharyngioma, Prader-Willi Syndrome, Frohlich's syndrome, Type II diabetics, GH-deficient subjects, normal variant short stature, Turner's syndrome, acute lymphoblastic leukemia, or other pathological conditions showing reduced metabolic activity or a decrease in resting energy expenditure as a percentage of total fat-free mass.

In some embodiments, including any of the foregoing embodiments, the subject experiences aberrant thermogenesis or aberrant adipose cell content or function. In some embodiments, the abnormal metabolism comprises misregulation of PGC-1 activity. In some further embodiments, the misregulation of PGC-1 activity comprises downregulation of PGC-1 activity. In other embodiments, the misregulation of PGC-1 activity comprises upregulation of PGC-1 activity.

In various embodiments, the improving the metabolic function comprises improving the functioning of mitochondria. In some embodiments, the metabolic function comprises one or more of: cellular proliferation, growth, differentiation, or migration; cellular regulation of homeostasis; inter- or intra-cellular communication; tissue function; and systemic response in an organism. In some embodiments, the tissue function comprises liver function, muscle function, or adipocyte function. In further embodiments, the systemic response in an organism comprises one or more of a hormonal response. In still further embodiments, the hormonal response is an insulin response.

In some embodiments of the present disclosure, the 15-PGDH inhibitor reduces or blocks enzymatic activity of 15-PGDH. In an embodiment, the abnormal metabolism comprises a metabolic disorder or an obesity-related disorder. In a further embodiment, the metabolic disorder is Metabolic syndrome (MS). In an embodiment, the metabolic disorder results in or causes obesity. In a further embodiment, the metabolic disorder is selected from the group comprising: overeating and bulimia, polycystic ovarian disease, craniopharyngioma, Prader-Willi Syndrome, Frohlich's syndrome, Type II diabetics, GH-deficient subjects, normal variant short stature, Turner's syndrome, acute lymphoblastic leukemia, and other pathological conditions showing reduced metabolic activity or a decrease in resting energy expenditure as a percentage of total fat-free mass.

In some embodiments, the metabolic disorder or obesity-related disorder is selected from the group consisting of: insulin resistant obesity; diabetes; hyperphagia; endocrine abnormalities; triglyceride storage disease; Bardet-Biedl syndrome; Lawrence-Moon syndrome; Prader-Labhard-Willi syndrome; anorexia; and cachexia. In an embodiment, the subject experiences aberrant thermogenesis or aberrant adipose cell content or function.

In certain embodiments, the abnormal metabolism comprises misregulation of Pgc1α activity. In further embodiments, the misregulation of Pgc1α activity comprises downregulation of Pgc1α activity. In other embodiments, the misregulation of Pgc1α activity comprises upregulation of Pgc1a activity.

In some embodiments, the improving the metabolic function comprises increasing mitochondrial biogenesis. In an embodiment, the metabolic function comprises the functioning of mitochondria. In an embodiment, the improving the metabolic function comprises increasing mitophagy. In an embodiment, the improving the metabolic function comprises enriching mitochondrial oxidative phosphorylation or ATP synthesis.

In any of the embodiments, the metabolic function may be selected from the group consisting of: cellular proliferation, growth, differentiation, or migration; cellular regulation of homeostasis; inter- or intra-cellular communication; tissue function; and systemic response in an organism. In further embodiments, the tissue function comprises liver function, muscle function, or adipocyte function. In some embodiments, the systemic response in an organism comprises a hormonal response. In an embodiment, the hormonal response comprises an insulin response.

In certain embodiments, the abnormal metabolism comprises a condition selected from the group consisting of: inherited mitochondrial diseases; MERRF; mitochondrial encephalopathy with lactic acidosis and stroke-like episodes (MELAS); LHON; DOA; Leigh syndrome; Kearns-Sayre syndrome; Friedreich's ataxia; other myopathies; cardiomyopathy; encephalomyopathy; renal tubular acidosis; neurodegenerative diseases; Parkinson's disease; Alzheimer's disease; ALS; motor neuron diseases; epilepsy; genetic diseases; Huntington's Disease; mood disorders; schizophrenia; bipolar disorder; and age-associated diseases.

In some embodiments, including any of the foregoing embodiments, the subject has one or more of a condition selected from the group consisting of: inherited mitochondrial diseases; MERRF; MELAS; LHON; DOA; Leigh syndrome; KSS; Friedreich's ataxia; other myopathies; cardiomyopathy; encephalomyopathy; renal tubular acidosis; neurodegenerative diseases; Parkinson's disease; Alzheimer's disease; ALS; motor neuron diseases; other neurological diseases; epilepsy; genetic diseases; Huntington's Disease; mood disorders; schizophrenia; bipolar disorder; and age-associated diseases.

Other objects, features, and advantages of the present disclosure will be apparent to one of skill in the art from the following detailed description and figures.

### BRIEF DESCRIPTION OF THE DRAWINGS

FIGS. 1A-1H. Boost in mitochondrial biogenesis and function is triggered by 15-PGDH inhibition in aged muscles. (FIG. 1A) Heatmap of mitochondrial genes. (FIG. 1B) Expression level of Pgc1α by qPCR (n=4 young, n=4 aged veh, n=3 aged SW033291 (SW)). (FIG. 1C) Relative quantification of mitochondrial DNA to nuclear DNA (n=4 per group). (FIG. 1D) Representative images and quantifications of intermyofibrillar (IMF) mitochondria numbers, their size, and myofibrillar width from transmission electron micrographs (TEM) of longitudinal sections of EDL muscles of young, aged vehicle and aged SW (n=3 mice per condition; total mitochondria quantified, n=1,066 young, n=1,350 aged vehicle and n=1,371 aged SW). Scale bar=2 μm. (FIG. 1E) Citrate synthase activity of Gastrocnemius muscles (n=3 young, n=4 aged vehicle and n=4 aged SW). (FIG. 1F) Representative TA cross-section stained for succinate dehydrogenase (SDH) (left). Quantification of SDH mean average intensity per fiber (n=4 mice per condition). (FIG. 1G) Representative images of mitochondrial membrane potential (TMRM staining) in isolated EDL myofibers from young, aged vehicle and SW treated mice (n=4 mice per condition; total number of fibers n=52 young, n=139 aged vehicle and n=89 aged SW). (FIG. 1H) Immunoblots of muscle lysates from: aged vehicle and SW treated mice together with colchicine, young mice without treatment, young mice treated with colchicine and young mice fasted and treated with colchicine. Quantification (right) (n=3 aged veh and SW). *P<0.05, **P<0.01, ***P<0.001, ****P<0.0001. ANOVA test with Tukey's test (FIG. 1D) or Fisher's LSD (FIGS. 1A, 1F, 1G) for multiple comparisons; Unpaired t-test (FIGS. 1C, 1E, 1H). Means±s.e.m.

FIGS. 2A-2C. PGE2 treatment leads to activation of CREB in muscles and 15-PGDH inhibition leads to increased mitochondrial biogenesis and function in aged muscles. (FIG. 2A) Immunoblots of muscle lysates from young (3 mo.) C57BL/6 mice injected with PGE2 i.m. after 0, 30 or 60 minutes. (FIG. 2B) Quantification of immunoblot in (FIG. 2A) (n=3 mice per timepoint). (FIG. 2B) Representative images of intermyofibrillar (IMF) mitochondria from transmission electron micrographs (TEM) of longitudinal sections of EDL muscles of young, aged vehicle and aged SW (n=3 mice per condition; total mitochondria quantified, n=1,066 young, n=1,350 aged vehicle and n=1,371 aged SW). Different magnifications are as shown: 1,500×, 3,000× and 10,000× with error bars respectively: 5 μm, 2 μm and 500 nm. (FIG. 2C) Citrate synthase activity of Quadriceps (Quad) muscles (n=13 aged vehicle and n=8 aged SW). *P<0.05, **P<0.01. ANOVA test with Fisher's LSD for multiple comparisons (FIG. 2B); unpaired t-test (FIG. 2C). Means±s.e.m.

### DETAILED DESCRIPTION

### 1. Introduction

Prostaglandin E2 (PGE2), also known as dinoprostone, has been employed in various clinical settings including to induce labor in women and to augment hematopoietic stem cell transplantation. PGE2 can be used as an anticoagulant and antithrombotic agent. The role of PGE2 as a lipid mediator that can resolve inflammation is also well known. Nonsteroidal anti-inflammatory drugs (NSAIDs), inhibitors of cyclooxygenase 1 (COX-1) and/or cyclooxygenase 2 (COX-2), suppress inflammation by inhibiting prostanoids, mainly via PGE2 biosynthesis. Prostaglandin D2 (PGD2) is a structural isomer of PGE2, with the 9-keto and 11-hydroxy group on PGE2 reversed on PGD2. PGD2 plays a role in a number of biological functions including vasoconstriction, inflammation, the regulation of body temperature during sleep, chemotaxis, and male sexual development. PGE2 and PGD2 are both synthesized from arachidonic acid by cyclooxygenases (COX) and by prostaglandin E synthase enzymes or prostaglandin D synthase enzymes, respectively. Levels of PGE2 and PGD2 are physiologically regulated by the enzyme 15-hydroxyprostaglandin dehydrogenase (15-PGDH), which catalyzes the conversion of the 15-OH group of both PGE2 and PGD2 to a 15-keto group.

The present disclosure is based, in part, on the discovery that inhibition of 15-PGDH promotes mitochondrial biogenesis and function. As such, the methods described herein are useful for treating diseases associated with dysfunctional mitochondria and/or increased oxidative stress. For example, such methods can be useful for treating metabolic diseases associated with mitophagy. Without being bound by the following theory, it is believed that elevated 15-PGDH levels in tissues in subjects with mitochondrial diseases lead to PGE2 and/or PGD2 degradation in these tissues and thus to lower levels of PGE2 and/or PGD2 and of PGE2 and/or PGD2 signaling, which has deleterious effects on tissue function. Inhibiting 15-PGDH in these tissues may restore or increase PGE2 and/or PGD2 levels in the tissues to promote mitochondrial biogenesis and function, leading to an increase in tissue function. Thus, the use of a 15-PGDH inhibitor in accordance with the present disclosure to inhibit 15-PGDH activity and/or reduce 15-PGDH levels in tissues with elevated 15-PGDH levels can reduce mitochondrial dysfunction and/or oxidative stress in these tissues and provide improved quality of life and outcomes for subjects with mitochondrial diseases.

Treating tissues having mitochondrial damage and/or oxidative stress with inhibitors of 15-PGDH may provide numerous advantages, such as that it can result in an elevation of mitochondrial biogenesis and function in the tissues (e.g., increased level of peroxisome proliferator-activated receptor gamma coactivator 1-alpha (Pgc1α or PCG-1α), increased mitochondrial DNA relative to nuclear DNA, increased number of mitochondria, improved mitochondrial morphology, increased mitochondrial enzymes involved in respiration, electron transport chain, and/or ATP generation, increased mitochondria complexes (complex I-V), increased mitophagy or autophagy leading to mitochondria turnover, and/or increased mitochondrial membrane potential), that the treatment can be localized to specific cell types that express elevated levels of 15-PGDH, that it provides the ability to restore endogenous levels of PGE2 and/or PGD2 to achieve levels of PGE2 and/or PGD2 present in non-diseased tissues, and that it provides the possibility of targeting 15-PGDH with molecules with relatively long half-lives or by using gene therapy, in order to provide sustained, systemic PGE2 benefits.

### 2. General

Practicing the methods disclosed herein utilizes routine techniques in the field of molecular biology. Basic texts disclosing the general methods of use described herein include Sambrook and Russell, Molecular Cloning. A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 0.2X. Thus, “about X” is intended to teach and provide written description support for a claim limitation of, e.g., “0.98X.”

The terms “prostaglandin E2”, “PGE2”, and “dinoprostone” refer to prostaglandin that can be synthesized from arachidonic acid via cyclooxygenase (COX) enzymes and terminal prostaglandin E synthases (PGES). PGE2 plays a role in a number of biological functions including vasodilation, inflammation, and modulation of sleep/wake cycles. Structural and functional information about PGE2 can be found, e.g., in the entry for “Dinoprostone” of PubChem: pubchem.ncbi.nlm.nih.gov/compound/Dinoprostone, the contents of which are herein incorporated by reference in their entirety.

The term “prostaglandin D2” or “PGD2” refers to prostaglandin that can be synthesized from arachidonic acid via cyclooxygenase (COX) enzymes and PGD2 synthases (PTDS). PGD2 is a structural isomer of PGE2, with the 9-keto and 11-hydroxy group on PGE2 reversed on PGD2. PGD2 plays a role in a number of biological functions including vasoconstriction, inflammation, the regulation of body temperature during sleep, chemotaxis, and male sexual development. Structural and functional information about PGD2 can be found, e.g., in the entry for “Prostaglandin D2” of PubChem: pubchem.ncbi.nlm.nih.gov/compound/448457, the contents of which are herein incorporated by reference in their entirety.

“15-PGDH” (15-hydroxyprostaglandin dehydrogenase) is an enzyme involved in the inactivation of a number of active prostaglandins, e.g., by catalyzing oxidation of PGE2 to 15-keto-prostaglandin E2 (15-keto-PGE2), or the oxidation of PGD2 to 15-keto-prostaglandin D2 (15-keto-PGD2). The human enzyme is encoded by the HPGD gene (Gene ID: 3248). The enzyme is a member of the short-chain nonmetalloenzyme alcohol dehydrogenase protein family. Multiple isoforms of the enzyme exist, e.g., in humans, any of which can be targeted using the present methods. For example, any of human isoforms 1-6 (e.g., GenBank Accession Nos. NP_000851.2, NP_001139288.1, NP_001243236.1, NP_001243234.1, NP_001243235.1, NP_001350503.1, NP_001243230.1) can be targeted, as can any isoform with 50%, 60%, 70%, 80%, 85%, 90%, 95%, or higher identity to the amino acid sequences of any of GenBank Accession Nos. NP_000851.2, NP_001139288.1, NP_001243236.1, NP_001243234.1, NP_001243235.1, NP_001350503.1, NP_001243230.1, or of any other 15-PGDH enzyme.

A “15-PGDH inhibitor” refers to any agent that is capable of inhibiting, reducing, decreasing, attenuating, abolishing, eliminating, slowing, and/or counteracting in any way any aspect of the expression, stability, and/or activity of 15-PGDH. A 15-PGDH inhibitor can, for example, reduce any aspect of the expression, e.g., transcription, RNA processing, RNA stability, and/or translation of a gene encoding 15-PGDH, e.g., the human HPGD gene, by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Similarly, a 15-PGDH inhibitor can, for example, reduce the activity, e.g., enzymatic activity, of a 15-PGDH enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. Further, a 15-PGDH inhibitor can, for example, reduce the stability of a 15-PGDH enzyme by, e.g., 10%, 15%, 20%, 25%, 30%, 35%, 40%, 45%, 50%, 55%, 60%, 65%, 70%, 75%, 80%, 85%, 90%, 95%, or more as compared to a control, e.g., in the absence of the inhibitor, in vitro or in vivo. A “15-PGDH inhibitor”, also referred to herein as a “15-PGDH agent” or a “15-PGDH compound,” can be any molecule, either naturally occurring or synthetic, e.g., peptide, protein, oligopeptide (e.g., from about 5 to about 25 amino acids in length, e.g., about 5, about 10, about 15, about 20, or about 25 amino acids in length), small molecule (e.g., an organic molecule having a molecular weight of less than about 2500 daltons, e.g., less than about 2000, less than about 1000, or less than about 500 daltons), antibody, nanobody, polysaccharide, lipid, fatty acid, inhibitory RNA (e.g., siRNA, shRNA, microRNA), modified RNA, polynucleotide, oligonucleotide, e.g., antisense oligonucleotide, aptamer, affimer, drug compound, or other compound.

The terms “expression” and “expressed” refer to the production of a transcriptional and/or translational product, e.g., of a nucleic acid sequence encoding a protein (e.g., 15-PGDH). In some embodiments, the term refers to the production of a transcriptional and/or translational product encoded by a gene (e.g., the human HPGD gene) or a portion thereof. The level of expression of a DNA molecule in a cell may be assessed on the basis of either the amount of corresponding mRNA that is present within the cell or the amount of protein encoded by that DNA produced by the cell.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term includes antibody fragments having the same antigen specificity, and fusion products thereof.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. Thus, the terms “variable heavy chain,” “V_{H}”, or “VH” refer to the variable region of an immunoglobulin heavy chain, including an Fv, scFv, dsFv or Fab; while the terms “variable light chain,” “V_{L}”, or “VL” refer to the variable region of an immunoglobulin light chain, including of an Fv, scFv, dsFv or Fab. Equivalent molecules include antigen binding proteins having the desired antigen specificity, derived, for example, by modifying an antibody fragment or by selection from a phage display library.

The terms “antigen-binding portion” and “antigen-binding fragment” are used interchangeably herein and refer to one or more fragments of an antibody that retains the ability to specifically bind to an antigen (e.g., a 15-PGDH protein). Examples of antibody-binding fragments include, but are not limited to, a Fab fragment (a monovalent fragment consisting of the VL, VH, CL, and CH1 domains), F(ab′)₂ fragment (a bivalent fragment comprising two Fab fragments linked by a disulfide bridge at the hinge region), a single chain Fv (scFv), a disulfide-linked Fv (dsFv), complementarity determining regions (CDRs), VL (light chain variable region), VH (heavy chain variable region), nanobodies, and any combination of those or any other functional portion of an immunoglobulin peptide capable of binding to target antigen (see, e.g., Fundamental Immunology (Paul ed., 4th ed. 2001).

The phrase “specifically binds” refers to a molecule (e.g., a 15-PGDH inhibitor such as a small molecule or antibody) that binds to a target with greater affinity, avidity, more readily, and/or with greater duration to that target in a sample than it binds to a non-target compound. In some embodiments, a molecule that specifically binds a target (e.g., 15-PGDH) binds to the target with at least 2-fold greater affinity than non-target compounds, e.g., at least 3-fold, at least 4-fold, at least 5-fold, at least 6-fold, at least 7-fold, at least 8-fold, at least 9-fold, at least 10-fold, at least 20-fold, at least 25-fold, at least 50-fold or greater affinity. For example, in some embodiments, a molecule that specifically binds to 15-PGDH typically binds to 15-PGDH with at least a 2-fold greater affinity than to a non-15-PGDH target.

The term “derivative,” in the context of a compound, includes but is not limited to, amide, ether, ester, amino, carboxyl, acetyl, and/or alcohol derivatives of a given compound.

The term “treating” or “treatment” refers to any one of the following: ameliorating one or more symptoms of a disease or condition; preventing the manifestation of such symptoms before they occur; slowing down or completely preventing the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

The term “administer,” “administering,” or “administration” refers to the methods that may be used to enable delivery of agents or compositions such as the compounds described herein to a desired site of biological action. These methods include, but are not limited to, parenteral administration (e.g., intravenous, subcutaneous, intraperitoneal, intramuscular, intra-arterial, intravascular, intracardiac, intrathecal, intranasal, intradermal, intravitreal, and the like), transmucosal injection, oral administration, administration as a suppository, and topical administration. In some cases, the administration is systemic administration (e.g., administration into the circulatory system such that multiple tissues and/or organs are treated or affected). In some cases, the administration is local administration (e.g., directly to the tissue or organ such that the tissue and/or organ is treated or affected). One skilled in the art will know of additional methods for administering a therapeutically effective amount of the compounds described herein.

The term “therapeutically effective amount” or “therapeutically effective dose” or “effective amount” refers to an amount of a compound (e.g., 15-PGDH inhibitor) that is sufficient to bring about a beneficial or desired clinical effect. A therapeutically effective amount or dose may be based on factors individual to each patient, including, but not limited to, the patient's age, size, type or extent of disease or condition, stage of the disease or condition, route of administration, the type or extent of supplemental therapy used, and/or ongoing disease process and type of treatment desired (e.g., aggressive vs. conventional treatment). Therapeutically effective amounts of a compound or composition (e.g., a 15-PGDH inhibitor), as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The term “pharmaceutical composition” as used herein refers to a composition comprising a compound (e.g., 15-PGDH inhibitor) as described herein and one or more pharmaceutically acceptable carriers and/or pharmaceutically acceptable excipients.

The term “pharmaceutically acceptable carrier” as used herein refers to a carrier or a diluent that does not cause significant irritation to an organism and does not abrogate the biological activity and properties of the administered compound.

The terms “subject,” “individual,” and “patient” are used interchangeably herein to refer to a vertebrate, preferably a mammal, more preferably a human. Mammals include, but are not limited to, murines, rats, simians, humans, farm animals or livestock for human consumption such as pigs, cattle, and ovines, as well as sport animals and pets. Subjects also include vertebrates such as fish and poultry.

The term “acute regimen”, in the context of administration of a compound, refers to a temporary or brief application of a compound to a subject, e.g., human subject, or to a repeated application of a compound to a subject, e.g., human subject, wherein a desired period of time (e.g., 1 day) lapses between applications. In some embodiments, an acute regimen includes an acute exposure (e.g., a single dose) of a compound to a subject over the course of treatment or over an extended period of time. In other embodiments, an acute regimen includes intermittent exposure (e.g., repeated doses) of a compound to a subject in which a desired period of time lapses between each exposure.

The term “chronic regimen,” in the context of administration of a compound, refers to a repeated, chronic application of a compound to a subject, e.g., human subject, over an extended period of time such that the amount or level of the compound is substantially constant over a selected time period. In some embodiments, a chronic regimen includes a continuous exposure of a compound to a subject over an extended period of time.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. In particular embodiments, modified RNA molecules are used, e.g., mRNA with certain chemical modifications to allow increased stability and/or translation when introduced into cells, as described in more detail below. It will be appreciated that any of the RNAs used in the present methods, including nucleic acid inhibitors such as siRNA or shRNA, can be used with chemical modifications to enhance, e.g., stability and/or potency. e.g., as described in Dar et al., Scientific Reports 6: article no. 20031 (2016), and as presented in the database accessible at crdd.osdd.net/servers/sirnamod/.

“Polypeptide”, “peptide”, and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used in herein, the terms “identical” or percent “identity”, in the context of describing two or more polynucleotide or amino acid sequences, refer to two or more sequences or specified subsequences that are the same. Two sequences that are “substantially identical” have at least about 60% identity, preferably at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters are used.

### 4. Methods of Promoting Mitochondrial Biogenesis and Function

In one aspect, a method is provided for increasing mitochondrial biogenesis and function in a tissue of a subject, the method comprising: administering to the subject an amount of a 15-PGDH inhibitor effective to inhibit 15-PGDH activity and/or reduce 15-PGDH levels in the subject, thereby increasing mitochondrial biogenesis and function in the tissue of the subject. The administration of the 15-PGDH inhibitor can be systemic or local, and can enhance any of a number of aspects of the tissue including enhancing function, physiological activity, endurance, performance on any assay for assessing tissue function, or any other measure of tissue function or health in the subject.

In some embodiments, the level of PGE2 present within the tissue may be increased (e.g., after treatment with a 15-PGDH inhibitor, e.g., according to methods provided herein) relative to the level present in the tissue prior to the treatment (e.g., with the 15-PGDH inhibitor). The PGE2 level in the tissue may be increased (e.g., by any method disclosed herein) by at least about 10% (e.g., at least about 15%, at least about 20%, at least about 25%, at least about 30%, at least about 40%, at least about 50%, at least about 60%, at least about 70%, or greater) relative to the level present in the tissue prior to the treatment (e.g., with the 15-PGDH inhibitor). In some embodiments, the subject has a mitochondrial disease or disorder and the level of PGE2 present within the tissue may be increased (e.g., after treatment with a 15-PGDH inhibitor, e.g., according to methods provided herein) to a level substantially similar to a level present in the same tissue of a subject not having the mitochondrial disease or disorder. The PGE2 level in the tissue may be increased (e.g., by any method disclosed herein) to a level within about 50% or less of a level present in the same tissue of a subject not having the mitochondrial disease or disorder (e.g., within about 40%, within about 35%, within about 30%, within about 25%, within about 20%, within about 15%, within about 10%, within about 5%, or within about 1%).

In some embodiments, the level of a PGE2 metabolite present within the tissue may be decreased (e.g., after treatment with a 15-PGDH inhibitor, e.g., according to methods provided herein) relative to the level present in the tissue prior to the treatment (e.g., with the 15-PGDH inhibitor). The PGE2 metabolite level in the tissue may be decreased (e.g., by any method disclosed herein) by at least about 10% (e.g., at least about 15%, at least about 20%, at least about 25%, at least about 30%, at least about 40%, at least about 50%, at least about 60%, at least about 70%, or greater) relative to the level present in the tissue prior to the treatment (e.g., with the 15-PGDH inhibitor). In some embodiments, the subject has a mitochondrial disease or disorder and the level of a PGE2 metabolite present within the tissue may be decreased (e.g., after treatment with a 15-PGDH inhibitor, e.g., according to methods provided herein) to a level substantially similar to a level present in the same tissue of a subject not having the mitochondrial disease or disorder. The PGE2 metabolite level in the tissue may be decreased (e.g., by any method disclosed herein) to a level within about 50% or less of a level present in the same tissue of a subject not having the mitochondrial disease or disorder (e.g., within about 40%, within about 35%, within about 30%, within about 25%, within about 20%, within about 15%, within about 10%, within about 5%, or within about 1%). The PGE2 metabolite may be 15-keto PGE2, 13,14-dihydro-15-keto PGE2, or both.

In some embodiments, the methods provided herein (e.g., after treatment with a 15-PGDH inhibitor) result in enhanced mitochondrial biogenesis and function in the tissue as demonstrated by an increased level of peroxisome proliferator-activated receptor gamma coactivator 1-alpha (Pgc1α) in the tissue, increased mitochondrial DNA relative to nuclear DNA in the tissue, increased number of mitochondria in the tissue, improved mitochondrial morphology in the tissue, increased mitochondrial complexes, enzymes involved in ATP generation, electron transport, and/or respiration in mitochondria present in the tissue, increased activity of complexes I, II, III, IV, and/or V of the electron transport chain in mitochondria present in the tissue, increased mitochondrial membrane potential in the tissue, increased mitophagy or autophagy leading to mitochondria turnover in the tissue, or any combination thereof. Mitochondrial biogenesis and function of the tissue may be enhanced (e.g., by any method disclosed herein) by at least about 10% (e.g., at least about 15%, at least about 20%, at least about 25%, at least about 30%, at least about 40%, at least about 50%, at least about 60%, at least about 70%, or greater) relative to the tissue prior to the treatment (e.g., with the 15-PGDH inhibitor). In some embodiments, the subject has a mitochondrial disease or disorder and mitochondrial biogenesis and function of the tissue may be enhanced (e.g., after treatment with a 15-PGDH inhibitor, e.g., according to methods provided herein) to a level substantially similar to a level present in the same tissue of a subject not having the mitochondrial disease or disorder. Mitochondrial biogenesis and function of the tissue may be enhanced (e.g., by any method disclosed herein) to a level within about 50% or less of a level present in the same tissue of a subject not having the mitochondrial disease or disorder (e.g., within about 40%, within about 35%, within about 30%, within about 25%, within about 20%, within about 15%, within about 10%, within about 5%, or within about 1%).

Techniques for assessment of mitochondrial biogenesis and function are known to those skilled in the art. For example, mRNA levels of peroxisome proliferator-activated receptor gamma coactivator 1-alpha (Pgc1α), the master regulator for mitochondrial biogenesis, can be assayed in the tissue. Mitochondrial copy number, e.g., as reflected by the mitochondrial DNA content relative to nuclear DNA content, can also be quantified in the tissue. Morphological assessment of mitochondria present in the tissue can be performed by transmission electron microscopy (TEM). Mitochondrial function can be assessed by analyzing any enzyme of the Krebs cycle such as citrate synthase activity, aconitase activity, isocitrate dehydrogenase activity, alpha-ketoglutarate dehydrogenase, succinyl-CoA synthetase activity, succinic dehydrogenase activity, fumarase activity, and malate dehydrogenase activity in mitochondria isolated from the tissue. Mitochondrial function can also be assessed by measuring the electron transport chain components by western blot of key proteins of mitochondrial complexes I-V, measurement of mitochondrial bioenergetics in isolated mitochondria can be assessed by high throughput respirometry, ATP production or by measurement of ATP synthase activity, cytochrome C oxidase activity, cytochrome c reductase activity, and NADH dehydrogenase activity. Changes in mitochondrial membrane potential can be measured in living cells of the tissue using flow cytometry, fluorescence plate reader, or fluorescence microscopy with the red-fluorescent probe TMRM (tetramethylrhodamine, methyl ester) or MitoTracker. Changes in mitochondrial fragmentation including levels of mitochondrial fusion components mitofusin 1 and 2 (Mfn1 and Mfn2), levels of optic atrophy 1 (OPA1), and levels of mitochondrial fission components Drp1, Fis1, can be assessed. Changes in mitophagy and autophagy which can lead to improved function due to mitochondria degradation can be assayed by assessing changes in mRNA and proteins including PTEN-induced putative kinase 1 (PINK1), PARKIN, BCL2/adenovirus E1B 19 kDa protein-interacting protein 3 (BNIP3), microtubule-associated protein light chain 3 (LC3) and lysosome-associated membrane protein-2 (LAMP-2), as well as mitophagic receptors NIP3-like protein X (NIX) and mitofusin2 (MFN2), autophagy related genes (e.g., Atg4, 5, 7, 8, 9, 13, 32), changes in the autophagy flux including assessment of autophagosome proteins p62 and/or Lc3b after colchicine and/or bafilomycin treatments of cells or mice.

In some embodiments, treatment (e.g., with a 15-PGDH inhibitor, e.g., according to methods provided herein) results in a reduction, decrease, attenuation, or inhibition of mitochondrial dysfunction in the tissue. Mitochondrial dysfunction includes features such as diminished mitochondrial biogenesis, altered membrane potential, and decrease in mitochondrial number and altered activities of oxidative proteins due to the accumulation of reactive oxygen species (ROS) in cells and tissues. The level of mitochondrial dysfunction in cells and tissues can be measured using any of the techniques described herein for assessment of mitochondrial biogenesis and function.

In some embodiments, treatment (e.g., with a 15-PGDH inhibitor, e.g., according to methods provided herein) results in a reduction, decrease, attenuation, or inhibition of oxidative stress in the tissue. Oxidative stress refers to the imbalance of two opposite and antagonistic forces, the production of ROS and antioxidants, in which the damaging effects of ROS are more powerful compared to the compensatory effect of antioxidants in cells and tissues. The level of oxidative stress in cells and tissues can be measured indirectly by measuring the level of DNA/RNA damage, lipid peroxidation, and/or protein oxidation/nitration, or measured directly by measuring the level of ROS.

In some embodiments, the 15-PGDH inhibitor reduces mitochondrial dysfunction and/or oxidative stress in the tissue by at least about 10% (e.g., at least about 15%, at least about 20%, at least about 25%, at least about 30%, at least about 40%, at least about 50%, at least about 60%, at least about 70%, or greater) relative to the cells and tissues prior to the treatment (e.g., with the 15-PGDH inhibitor). In some embodiments, the subject has a mitochondrial disease and the 15-PGDH inhibitor reduces mitochondrial dysfunction and/or oxidative stress in the tissue to a level substantially similar to a level present in the same tissue of a subject not having the mitochondrial disease. Mitochondrial dysfunction and/or oxidative stress of the tissue may be reduced (e.g., by any method disclosed herein) to a level within about 50% or less of a level present in the same tissue of a subject not having the mitochondrial disease (e.g., within about 40%, within about 35%, within about 30%, within about 25%, within about 20%, within about 15%, within about 10%, within about 5%, or within about 1%).

The present disclosure also provides methods of administering the 15-PGDH inhibitor to treat a mitochondrial disorder, disease or condition. Such disorders, diseases, and conditions can include, for example, inherited mitochondrial diseases; myoclonic epilepsy with ragged red fibers (MERRF); mitochondrial encephalopathy with lactic acidosis and stroke-like episodes (MELAS); Leber hereditary optic neuropathy (LHON); dominant optic atrophy (DOA); Leigh syndrome; Kearns-Sayre syndrome (KSS); Friedreich's ataxia (FRDA); other myopathies; cardiomyopathy; encephalomyopathy; renal tubular acidosis; Parkinson's disease; Alzheimer's disease; amyotrophic lateral sclerosis (ALS); Huntington's Disease; developmental pervasive disorders or hearing loss.

In other embodiments, including any of the foregoing embodiments, the mitochondrial disorder, disease, or condition is selected from the group consisting of inherited mitochondrial diseases; myoclonic epilepsy with ragged red fibers (MERRF); mitochondrial encephalopathy with lactic acidosis and stroke-like episodes (MELAS); Leber hereditary optic neuropathy (LHON); dominant optic atrophy (DOA); Leigh syndrome; Kearns-Sayre syndrome (KSS); Friedreich's ataxia (FRDA); other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; other neurological diseases; epilepsy; genetic diseases; Huntington's Disease; mood disorders; schizophrenia; bipolar disorder; and age-associated diseases.

In any of the methods above for modulating one or more energy biomarkers, normalizing one or more energy biomarkers, or enhancing one or more energy biomarkers, the energy biomarker can be selected from the group consisting of: lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H+) levels; NADPH (NADPH+H+) levels; NAD levels; NADP levels; ATP levels; reduced coenzyme Q (CoQred) levels; oxidized coenzyme Q (CoQox) levels; total coenzyme Q (CoQlot) levels; oxidized cytochrome C levels; reduced cytochrome C levels; oxidized cytochrome C/reduced cytochrome C ratio; acetoacetate levels; beta-hydroxy butyrate levels; acetoacetate/beta-hydroxy butyrate ratio; 8-hydroxy-2′-deoxyguanosine (8-OHdG) levels; levels of reactive oxygen species; levels of oxygen consumption (VO2); levels of carbon dioxide output (VCO2); respiratory quotient (VCO2/VO2); exercise tolerance; and anaerobic threshold.

The present disclosure also provides methods of measuring 15-PGDH levels in tissues of a subject with a mitochondrial disease, disorder, or condition. Such methods are useful, e.g., for the use of 15-PGDH as a biomarker of mitochondrial disease and/or for a loss or decrease of mitochondrial function of tissues, e.g., wherein an elevated level of 15-PGDH levels or activity, e.g., an increase of at least about 10%, at least about 20%, at least about 30%, at least about 40%, at least about 50%, at least about 60%, at least about 70%, at least about 80%, at least about 90%, about 100% relative to a control level in a subject without a mitochondrial disease, disorder, or condition is indicative of mitochondrial disease, disorder, condition or a loss or decrease of function in the tissue. In such methods, 15-PGDH can be assessed in any of a number of ways, e.g., by detecting levels of a transcript encoding a 15-PGDH protein, by detecting levels of a 15-PGDH polypeptide, or by detecting 15-PGDH enzymatic activity.

In particular embodiments, the inhibition of 15-PGDH in the subject leads to an increase in PGE2 and/or PGD2, e.g., an elevation, increase, or restoration of PGE2 and/or PGD2 levels, in the tissue of the subject, and a decrease in PGE2 and/or PGD2 metabolites such as 15-keto-PGE2, 13,14-dihydro-15-keto-PGE2 (PGEM), 15-keto-PGD2, and 13,14-dihydro-15-keto-PGD2. In some embodiments, the inhibition also leads to increased signaling through PGE2 receptors, e.g., EP1, EP2, EP3, and/or EP4 (also known as Ptger1, Ptger2, Ptger3, Ptger4) in the tissue. In some embodiments, the inhibition also leads to increased signaling through PGD2 receptors, e.g., DP1 and/or DP2 (also known as PTGDR1, PTGDR2/CRTH2).

In particular embodiments, the herein-described benefits of 15-PGDH inhibitor administration in the tissue, e.g., enhanced mitochondrial biogenesis and function, etc., occur independently of any regeneration of the tissue in the subject. In other words, while there may be regeneration of the tissue in the subject, e.g., if the tissue has been injured or damaged, the herein-described effects do not require the regeneration and would occur even without the regeneration. In particular embodiments, the tissue is not injured or damaged and has not or does not undergo regeneration.

### Subjects

The subject can be any subject, e.g., a human or other mammal, with a mitochondrial disease or at risk of having a mitochondrial disease. In some embodiments, the subject is a human. In some embodiments, the subject is an adult. In some embodiments, the subject is a child. In some embodiments, the subject is female (e.g., an adult female). In some embodiments, the subject is male (e.g., an adult male).

In any of the above embodiments, the subject can be selected from the group consisting of: a subject with a mitochondrial disease; a subject undergoing strenuous or prolonged physical activity; a subject with chronic energy problems; a subject with chronic respiratory problems; a pregnant female; a pregnant female in labor; a neonate; a premature neonate; a subject exposed to an extreme environment; a subject exposed to a hot environment; a subject exposed to a cold environment; a subject exposed to an environment with lower-than-average oxygen content; a subject exposed to an environment with higher-than-average carbon dioxide content; a subject exposed to an environment with higher-than-average levels of air pollution; a subject with lung disease; a subject with lower-than-average lung capacity; a tubercular patient; a lung cancer patient; an emphysema patient; a cystic fibrosis patient; a subject recovering from surgery; a subject recovering from illness; a subject undergoing acute trauma; a subject in shock; a subject requiring acute oxygen administration; a subject requiring chronic oxygen administration; an elderly subject; an elderly subject experiencing decreased energy; and a subject suffering from chronic fatigue; subjects suffering from chronic fatigue syndrome; subjects undergoing acute trauma; subjects in shock; subjects requiring acute oxygen administration; subjects requiring chronic oxygen administration; or other subjects with acute, chronic, or ongoing energy demands who can benefit from enhancement of energy biomarkers.

In some embodiments, the subject is human, and the method further comprises a step in which the human is selected for treatment with the 15-PGDH inhibitor based on a diagnosis of a mitochondrial disease, or on the potential for or risk of developing a mitochondrial disease. In some such embodiments, the human is selected based on his or her age. For example, a human can be selected for treatment based on age who is over 20, 25, 30, 35, 40, 45, 50, 55, 60, 65, 70, 75, 80, 85, 90, 95, or 100 years old or older, or any age in which the human has or potentially has a mitochondrial disease. In some embodiments, the human is selected based on a potential for a mitochondrial disease, based on the presence or potential presence of an environmental, lifestyle, or medical factor linked to mitochondrial dysfunction and/or oxidative stress in tissues, such as smoking, drinking, diet, lack of physical activity, insufficient sleep, drug use, exposure to UV rays, exposure to extreme temperatures, stress, excess weight, or health-related factors such as infections, mental illness, cancer, diabetes, etc.

In some embodiments, the subject is determined to have a mitochondrial disease as determined using any method of assessing any measure of the function, performance, health, strength, endurance, physiological activity, or any other property of a tissue, e.g., a performance-based, imaging-based, physiological, molecular, cellular, or functional assay. In some embodiments, the subject is selected for treatment based on an assessment of mitochondrial biogenesis and function in a tissue. In some embodiments, the subject is selected for treatment based on a detection of elevated levels of 15-PGDH transcript, protein, or enzymatic activity in a tissue, or on a detection of decreased levels of PGE2 and/or PGD2 in the tissue.

In some embodiments, the methods comprise an additional step subsequent to the administration of a 15-PGDH inhibitor, comprising assessing the health, function, performance, or any other property of a tissue in the subject, or comprising assessing the level of 15-PGDH (e.g., of 15-PGDH protein, transcript, or activity) and/or PGE2 and/or PGD2 in the tissue in the subject, e.g., to ascertain the potential effects of the prior administration of the 15-PGDH inhibitor on the tissue. In some such embodiments, the health, function, performance, 15-PGDH level, PGE2 level, PGD2 level, or other property of the tissue is detected or examined and compared to the health, function, performance, 15-PGDH level, PGE2 level, PGD2 level, or other property of the tissue prior to the administration of the 15-PGDH inhibitor or to a control value, wherein a determination that the health, function, or performance of the tissue has improved, that the 15-PGDH level has decreased, that the PGE2 level and/or PGD2 level has increased, in the tissue subsequent to the administration of the inhibitor as compared to the value obtained prior to the administration of the 15-PGDH inhibitor or relative to a control value, indicates that the 15-PGDH inhibitor has had a beneficial effect in the tissue of the subject.

In some embodiments, the subject has a mitochondrial disease such as a metabolic disease (e.g., diabetes mellitus type 2, obesity, dyslipidemia, fatty liver disease), a neurodegenerative disease (e.g., Alzheimer's disease, Huntington's disease, Parkinson's disease), cancer, a motor neuron disease (e.g., spinal muscular atrophy, amyotrophic lateral sclerosis, myasthenia gravis, bulbar palsy, Friedreich's ataxia), a cardiovascular disease (e.g., cardiomyopathies, atherosclerosis), a neurobehavioral or psychiatric disease (e.g., autism spectrum disorders, schizophrenia, bipolar and mood disorders, migraine), an autoimmune disease (e.g., multiple sclerosis, systemic lupus erythematosus, type 1 diabetes), a chronic kidney disease, mitochondrial encephalopathy lactic acidosis and stroke-like episodes (MELAS), chronic obstructive pulmonary disease (COPD), ventilator induced diaphragmatic dysfunction (VIDD), myalgic encephalomyelitis/chronic fatigue syndrome, Leber hereditary optic neuropathy (LHON), chemotherapy-induced peripheral neuropathy, and the like.

In some embodiments, the subject has a metabolic disorder or an obesity-related disorder. As used herein, these terms may include a disorder, disease or condition which is caused or characterized by an abnormal metabolism in a subject. Metabolic disorders include diseases, disorders, or conditions associated with aberrant thermogenesis or aberrant adipose cell (e.g., brown or white adipose cell) content or function. Metabolic disorders can be characterized by a misregulation (e.g., downregulation or upregulation) of Pgc1α activity. Metabolic disorders can detrimentally affect cellular functions such as cellular proliferation, growth, differentiation, or migration, cellular regulation of homeostasis, inter- or intra-cellular communication; tissue function, such as liver function, muscle function, or adipocyte function; systemic responses in an organism, such as hormonal responses (e.g., insulin response). Examples of metabolic or obesity-related disorders include obesity, including insulin resistant obesity, diabetes, hyperphagia, endocrine abnormalities, triglyceride storage disease. Bardet-Biedl syndrome, Lawrence-Moon syndrome, Prader-Labhart-Willi syndrome, anorexia, and cachexia. The obesity described herein may be due to any cause, whether genetic or environmental. Examples of disorders that may result in obesity or be the cause of obesity include overeating and bulimia, polycystic ovarian disease, craniopharyngioma, Prader-Willi Syndrome, Frohlich's syndrome, Type II diabetics, GH-deficient subjects, normal variant short stature, Turner's syndrome, and other pathological conditions showing reduced metabolic activity or a decrease in resting energy expenditure as a percentage of total fat-free mass, e.g., children with acute lymphoblastic leukemia.

In another aspect of the present disclosure, a method for treating Metabolic syndrome is disclosed. Metabolic syndrome (MS) describes a cluster of metabolic abnormalities including obesity, insulin resistance, hypertension and dyslipidemia. Inflammation is associated with oxidative stress which is one obesity-related feature participating in the development of MS. Oxidative stress results from excess of reactive oxygen species (ROS) production overwhelming antioxidant defenses. ROS are mainly produced as by-products of the mitochondrial electron transport chain involved in ATP production (oxidative phosphorylation). Excess fatty acids and glucose are known to be deleterious for mitochondrial function, thus increasing ROS production. ROS can oxidize cell macromolecules, leading to impaired cellular homeostasis and associated pathologies such as cancer.

The present disclosure also provides methods for treating diseases resulting from under expression or overexpression of certain genes. For example, studies on mice with TRx2 deficiencies suggest that Trx2 deficiency causes mitochondrial structure damage and excessive mitophagy, which contributes to mitochondrial dysfunction and loss, resulting in disrupted metabolic homeostasis in adipose tissue. Feng et al., Mitophagy-mediated adipose inflammation contributes to type 2 diabetes with hepatic insulin resistance. J Exp. Med., 2021; 218(3): e20201416. Trx2 KO mice developed hepatic insulin resistance and hyperglycemia. Trx2 deficiency induced excessive mitophagy and mitochondrial structural damage, which contributed to adipose mitochondrial loss and dysfunction and further disrupted whole-body metabolic homeostasis. Mechanistically, Trx2 deficiency via ROS generation promoted NF-κB-dependent accumulation of autophagy receptor p62/SQSTM1 to target damaged mitochondria for autophagic removal. The findings suggest the potentiality of blocking inflammation activation in the treatment of hepatic insulin resistance and type II diabetes mellitus associated with adipose mitophagy. Thus, Trx2 is a critical hub for integrating oxidative stress, inflammation, and mitochondrial metabolism in adipose tissue, which reveals that adipose mitophagy links NF-κB signal activation to hepatic insulin resistance and type II diabetes mellitus. The 15-PGDH inhibitors of the present disclosure may thus help mediate mitophagy and ameliorate adipose metabolic disorders and progression of type II diabetes.

Another aspect of the present disclosure pertains to methods for treating a subject, having a disease or disorder characterized by (or associated with) aberrant or abnormal Pgc1α (also referred to herein as “PGC-1α”) nucleic acid expression and/or PGC-1α protein activity. These methods include the step of administering a PGC-1α modulator to the subject such that treatment occurs. The language “aberrant or abnormal PGC-1α expression” refers to expression of a non-wild-type PGC-1α protein or a non-wild-type level of expression of a PGC-1α protein. Aberrant or abnormal PGC-1α protein activity refers to a non-wild-type PGC-1α protein activity or a non-wild-type level of PGC-1α protein activity. As the PGC-1α protein is involved in, for example, a pathway involving type I muscle formation, aberrant or abnormal PGC-1α protein activity or nucleic acid expression interferes with the normal expression of type I muscle specific genes and/or type I muscle differentiation.

Non-limiting examples of disorders or diseases characterized by or associated with abnormal or aberrant PGC-1α protein activity or nucleic acid expression (also referred to herein as PGC-1α associated disorders or as type I muscle associated disorders) include cardiovascular disorders (e.g., heart failure), disuse atrophy, muscle wasting (e.g., caused by disorders such as cancer, AIDS, or other infectious diseases), mitochondrial myopathies, systemic metabolic disorders (e.g., diabetes, insulin resistance, hypoglycemia, obesity, body weight disorders, cachexia, or anorexia). See, e.g., Braunwald et al., eds. Harrison's Principles of Internal Medicine, Eleventh Edition (McGraw-Hill Book Company, New York, 1987) pp. 1778-1797; Robbins. S. L. et al., Pathologic Basis of Disease, 3rd Edition (W.B. Saunders Company, Philadelphia, 1984) p. 972 for further descriptions of such disorders.

The present methods can be used to treat any tissue, or organs including such tissues, or cells within such tissues, including skeletal muscle, epithelial tissue, nerve tissue, connective tissue, smooth muscle, cardiac muscle, epidermal tissues, vascular tissues, heart, kidney, brain, bone, cartilage, brown fat, spleen, liver, colon, sensory organs, thyroid, lung, blood, small intestine, dental tissue, ovaries or other reproductive tissue, hair, cochlea, oligodendrocytes, etc.

### 5. Assessing 15-PGDH Levels

Any of a number of methods can be used to assess the level of 15-PGDH in a tissue, e.g., when using 15-PGDH as a biomarker or when assessing the efficacy of an inhibitor of 15-PGDH. For example, the level of 15-PGDH can be assessed by examining the transcription of a gene encoding 15-PGDH (e.g., the Hpgd gene), by examining the levels of 15-PGDH protein in the tissue, or by measuring the 15-PGDH enzyme activity in the tissue. Such methods can be performed on the overall tissue or on a subset of cells within the tissue.

In some embodiments, the methods involve the measurement of 15-PGDH enzyme activity, e.g., using standard methods such as incubating a candidate compound in the presence of 15-PGDH enzyme, NAD(+), and PGE2 in an appropriate reaction buffer, and monitoring the generation of NADH (see, e.g., Zhang et al., (2015) Science 348: 1224), or by using any of a number of available kits such as the fluorometric PicoProbe 15-PGDH Activity Assay Kit (BioVision), or by using any of the methods and/or indices described in, e.g., EP 2838533 B1.

In some embodiments, the methods involve the detection of 15-PGDH-encoding polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See. e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g. TaqMan® Gene Expression Assays, ThermoFisher Scientific).

In some embodiments, the methods involve the detection of 15-PGDH protein expression or stability, e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

### 6. 15-PGDH as a Biomarker

In some embodiments, 15-PGDH may be used as a biomarker for mitochondrial damage and/or oxidative stress in a tissue, or for the presence or potential for a mitochondrial disease. For example, a detection of an increase in 15-PGDH levels in a tissue, e.g., in the overall tissue or in specific cells within the tissue, is indicative of mitochondrial damage and/or oxidative stress in the tissue, of a loss or decrease of function or health of the tissue related to mitochondrial damage and/or oxidative stress, or of the presence of a mitochondrial disease. For example, a detected increase of about 10%, about 20%, about 30%, about 40%, about 50%, about 60%, about 70%, about 80%, about 90%, about 100%, or more 15-PGDH in a tissue as compared to in a control tissue from a subject without a mitochondrial disease may be indicative of mitochondrial damage and/or oxidative stress in the tissue, of a loss or decrease of function or health of the tissue related to mitochondrial damage and/or oxidative stress, or of the presence of a mitochondrial disease.

### 7. 15-PGDH Inhibitors

Any agent that reduces, decreases, counteracts, attenuates, inhibits, blocks, downregulates, or eliminates in any way the expression, stability or activity, e.g., enzymatic activity, of 15-PGDH can be used in the present methods. Inhibitors can be small molecule compounds, peptides, polypeptides, nucleic acids, antibodies. e.g., blocking antibodies or nanobodies, or any other molecule that reduces, decreases, counteracts, attenuates, inhibits, blocks, downregulates, or eliminates in any way the expression, stability, and/or activity of 15-PGDH, e.g., the enzymatic activity of 15-PGDH.

In some embodiments, the 15-PGDH inhibitor decreases the activity, stability, or expression of 15-PGDH by at least about 10%, about 15%, about 20%, about 25%, about 30%, about 35%, about 40%, about 45%, about 50%, about 55%, about 60%, about 65%, about 70%, about 75%, about 80%, about 85%, about 90%, or more relative to a control level, e.g., in the absence of the inhibitor, in vivo or in vitro.

The efficacy of inhibitors can be assessed, e.g., by measuring 15-PGDH enzyme activity, e.g., using standard methods such as incubating a candidate compound in the presence of 15-PGDH enzyme, NAD(+), and PGE2 in an appropriate reaction buffer, and monitoring the generation of NADH (see. e.g., Zhang et al., (2015) Science 348: 1224), or by using any of a number of available kits such as the fluorometric PicoProbe 15-PGDH Activity Assay Kit (BioVision), or by using any of the methods and/or indices described in, e.g., EP 2838533 B1.

The efficacy of inhibitors can also be assessed, e.g., by detection of decreased polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays. ThermoFisher Scientific).

In some embodiments, the 15-PGDH inhibitor is considered effective if the level of expression of a 15-PGDH-encoding polynucleotide is decreased by at least about 10%, at least about 20%, at least about 30%, at least about 40%, at least about 50%, at least about 60%, at least about 70%, at least about 80%, at least about 90% or more as compared to the reference value, e.g., the value in the absence of the inhibitor, in vitro or in vivo. In some embodiments, a 15-PGDH inhibitor is considered effective if the level of expression of a 15-PGDH-encoding polynucleotide is decreased by at least 1.5-fold, at least 2-fold, at least 3-fold, at least 4-fold, at least 5-fold, at least 6-fold, at least 7-fold, at least 8-fold, at least 9-fold, at least 10-fold or more as compared to the reference value.

The effectiveness of a 15-PGDH inhibitor can also be assessed by detecting protein expression or stability, e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed Sci., 699:463-80 (1997)).

For determining whether 15-PGDH protein levels are decreased in the presence of a 15-PGDH inhibitor, the method comprises comparing the level of the protein (e.g., 15-PGDH protein) in the presence of the inhibitor to a reference value, e.g., the level in the absence of the inhibitor. In some embodiments, a 15-PGDH protein is decreased in the presence of an inhibitor if the level of the 15-PGDH protein is decreased by at least about 10%, at least about 20%, at least about 30%, at least about 40%, at least about 50%, at least about 60%, at least about 70%, at least about 80%, at least about 90% or more as compared to the reference value. In some embodiments, a 15-PGDH protein is decreased in the presence of an inhibitor if the level of the 15-PGDH protein is decreased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold or more as compared to the reference value.

### Small Molecules

In particular embodiments, 15-PGDH is inhibited by the administration of a small molecule inhibitor. Any small molecule inhibitor can be used that reduces, e.g., by about 10%, about 15%, about 20%, about 25%, about 30%, about 35%, about 40%, about 45%, about 50%, about 55%, about 60%, about 65%, about 70%, about 75%, about 80%, about 85%, about 90%, or more, the expression, stability, or activity of 15-PGDH relative to a control, e.g., the expression, stability, or activity in the absence of the inhibitor. In particular embodiments, small molecule inhibitors may be used that can reduce the enzymatic activity of 15-PGDH in vitro or in vivo. Non-limiting examples of small molecule compounds that can be used in the present methods include the small molecules disclosed in EP 2838533 B1, the entire disclosure of which is herein incorporated by reference. Small molecules can include, inter alia, the small molecules disclosed in Table 2 of EP 2838533 B1, i.e., SW033291, SW033291 isomer B. SW033291 isomer A, SW033292, 413423, 980653, 405320, SW208078, SW208079, SW033290, SW208080, SW208081, SW206976, SW206977, SW206978, SW206979, SW206980, SW206992, SW208064, SW208065, SW208066, SW208067, SW208068, SW208069, SW208070, as well as combinations, derivatives, isomers, or tautomers thereof. In particular embodiments, the 15-PGDH inhibitor used is SW033291 (2-(butylsulfinyl)-4-phenyl-6-(thiophen-2-yl)thieno[2,3-b]pyridin-3-amine; PubChem CID: 3337839).

In some embodiments, the 15-PGDH inhibitor is a thiazolidinedione derivative (e.g., benzylidenethiazolidine-2,4-dione derivative) such as (5-(4-(2-(thiophen-2-yl)ethoxy)benzylidene)thiazolidine-2,4-dione), 5-(3-chloro-4-phenylethoxybenzylidene)thiazolidine-2,4-dione, 5-(4-(2-cyclohexylethoxy)benzylidene)thiazolidine-2,4-dione, 5-(3-chloro-4-(2-cyclohexylethoxy)benzyl)thiazolidine-2,4-dione, (Z)-N-benzyl-4-((2,4-dioxothiazolidin-5-ylidene)methyl)benzamide, or any of the compounds disclosed in Choi et al. (2013) Bioorganic & Medicinal Chemistry 21:4477-4484; Wu et al. (2010) Bioorg. Med. Chem. 18(2010) 1428-1433; Wu et al. (2011) J. Med. Chem. 54:5260-5264; or Yu et al. (2019) Biotechnology and Bioprocess Engineering 24:464475, the entire disclosures of which are herein incorporated by reference. In some embodiments, the 15-PGDH inhibitor is a COX inhibitor or chemopreventive agent such as ciglitazone (CID: 2750), or any of the compounds disclosed in Cho et al. (2002) Prostaglandins, Leukotrienes and Essential Fatty Acids 67(6):461-465, the entire disclosure of which is herein incorporated by reference.

In some embodiments, the 15-PGDH inhibitor is a compound containing a benzimidazole group, such as (1-(4-methoxyphenyl)-1H-benzo[d]imidazol-5-yl)(piperidin-1-yl)methanone (CID: 3474778), or a compound containing a triazole group, such as 3-(2,5-dimethyl-1-(p-tolyl)-1H-pyrrol-3-yl)-6,7,8,9-tetrahydro-5H-[1,2,4]triazolo[4,3-a]azepine (CID: 71307851), or any of the compounds disclosed in Duveau et al. (2015) (“Discovery of two small molecule inhibitors, ML387 and ML388, of human NAD+-dependent 15-hydroxyprostaglandin dehydrogenase,” published in Probe Reports from the NIH Molecular Libraries Program [Internet]), the entire disclosure of which is herein incorporated by reference. In some embodiments, the 15-PGDH inhibitor is 1-(3-methylphenyl)-1H-benzimidazol-5-yl)(piperidin-1-yl)methanone (CID: 4249877) or any of the compounds disclosed in Niesen et al. (2010) PLoS ONE 5(11):e13719, the entire disclosure of which is herein incorporated by reference. In some embodiments, the 15-PGDH inhibitor is 2-((6-bromo-4H-imidazo[4,5-b]pyridin-2-ylthio)methyl)benzonitrile (CID: 3245059), piperidin-1-yl(1-m-tolyl-1H-benzo[d]imidazol-5-yl)methanone (CID: 3243760), or 3-(2,5-dimethyl-1-phenyl-1H-pyrrol-3-yl)-6,7,8,9-tetrahydro-5H-[1,2,4]triazolo[4,3-a]azepine (CID: 2331284), or any of the compounds disclosed in Jadhav et al. (2011) (“Potent and selective inhibitors of NAD+-dependent 15-hydroxyprostaglandin dehydrogenase (HPGD),” published in Probe Reports from the NIH Molecular Libraries Program [Internet]), the entire disclosure of which is herein incorporated by reference.

In some embodiments, the 15-PGDH inhibitor is TD88 or any of the compounds disclosed in Sco et al. (2015) Prostaglandins, Leukotrienes and Essential Fatty Acids 97:35-41, or Shao et al. (2015) Genes & Diseases 2(4):295-298, the entire disclosures of which are herein incorporated by reference. In some embodiments, the 15-PGDH inhibitor is EEAH (Ethanol extract of Artocarpus heterophyllus) or any of the compounds disclosed in Karna (2017) Pharmacogn Mag. 2017 January; 13(Suppl 1): S122-S126, the entire disclosure of which is herein incorporated by reference.

### Inhibitory Nucleic Acids

In some embodiments, the agent comprises an inhibitory nucleic acid, e.g., antisense DNA or RNA, small interfering RNA (siRNA), microRNA (miRNA), or short hairpin RNA (shRNA). In some embodiments, the inhibitory RNA targets a sequence that is identical or substantially identical (e.g., at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical) to a target sequence in a 15-PGDH polynucleotide (e.g., a portion comprising at least about 20, at least about 30, at least about 40, at least about 50, at least about 60, at least about 70, at least about 80, at least about 90, or at least about 100 contiguous nucleotides, e.g., from about 20-500, about 20-250, about 20-100, about 50-500, or about 50-250 contiguous nucleotides of a 15-PGDH-encoding polynucleotide sequence (e.g., the human HPGD gene, Gene ID: 3248, including of any of its transcript variants, e.g., as set forth in GenBank Accession Nos. NM_000860.6, NM_001145816.2, NM_001256301.1, NM_001256305.1, NM_001256306.1, NM_001256307.1, or NM_001363574.1).

In some embodiments, the methods described herein comprise treating a subject, e.g., a subject with a disease associated with mitochondrial dysfunction and/or oxidative stress such as a mitochondrial disease, using an shRNA or siRNA. A shRNA is an artificial RNA molecule with a hairpin turn that can be used to silence target gene expression via the siRNA it produces in cells. See. e.g., Fire et. al., Nature 391:806-811, 1998; Elbashir et al., Nature 411:494-498, 2001; Chakraborty et al., Mol Ther Nucleic Acids 8:132-143, 2017; and Bouard et al., Br. J. Pharmacol. 157:153-165, 2009. In some embodiments, a method of treating a subject, e.g., with a disease associated with mitochondrial dysfunction and/or oxidative stress such as a mitochondrial disease, comprises administering to the subject a therapeutically effective amount of a modified RNA or a vector comprising a polynucleotide that encodes an shRNA or siRNA capable of hybridizing to a portion of a 15-PGDH mRNA (e.g., a portion of the human 15-PGDH-encoding polynucleotide sequence set forth in any of GenBank Accession Nos. NM_000860.6, NM_001145816.2, NM_001256301.1, NM_001256305.1, NM_001256306.1, NM_001256307.1, or NM_001363574.1). In some embodiments, the vector further comprises appropriate expression control elements known in the art, including, e.g., promoters (e.g., inducible promoters or tissue specific promoters), enhancers, and transcription terminators.

In some embodiments, the agent is a 15-PGDH-specific microRNA (miRNA or miR). A microRNA is a small non-coding RNA molecule that functions in RNA silencing and post-transcriptional regulation of gene expression. miRNAs base pair with complementary sequences within the mRNA transcript. As a result, the mRNA transcript may be silenced by one or more of the mechanisms such as cleavage of the mRNA strand, destabilization of the mRNA through shortening of its poly(A) tail, and decrease in the translation efficiency of the mRNA transcript into proteins by ribosomes.

In some embodiments, the agent may be an antisense oligonucleotide, e.g., an RNase H-dependent antisense oligonucleotide (ASO). ASOs are single-stranded, chemically modified oligonucleotides that bind to complementary sequences in target mRNAs and reduce gene expression both by RNase H-mediated cleavage of the target RNA and by inhibition of translation by steric blockade of ribosomes. In some embodiments, the oligonucleotide is capable of hybridizing to a portion of a 15-PGDH mRNA (e.g., a portion of a human 15-PGDH-encoding polynucleotide sequence as set forth in any of GenBank Accession Nos. NM_000860.6, NM_001145816.2, NM_001256301.1, NM_001256305.1, NM_001256306.1, NM_001256307.1, or NM_001363574.1). In some embodiments, the oligonucleotide has a length of about 10-30 nucleotides (e.g., about 10, 12, 14, 16, 18, 20, 22, 24, 26, 28, or 30 nucleotides). In some embodiments, the oligonucleotide has 100% complementarity to the portion of the mRNA transcript it binds. In other embodiments, the DNA oligonucleotide has less than 100% complementarity (e.g., about 95%, about 90%, about 85%, about 80%, about 75%, or about 70% complementarity) to the portion of the mRNA transcript it binds, but can still form a stable RNA:DNA duplex for the RNase H to cleave the mRNA transcript.

Suitable antisense molecules, siRNA, miRNA, and shRNA can be produced by standard methods of oligonucleotide synthesis or by ordering such molecules from a contract research organization or supplier by providing the polynucleotide sequence being targeted. The manufacture and deployment of such antisense molecules in general terms may be accomplished using standard techniques described in contemporary reference texts: for example, Gene and Cell Therapy: Therapeutic Mechanisms and Strategies. 4^{th} edition by N. S. Templeton; Translating Gene Therapy to the Clinic: Techniques and Approaches. 1^{st} edition by J. Laurence and M. Franklin; High-Throughput RNAi Screening: Methods and Protocols (Methods in Molecular Biology) by D. O. Azorsa and S. Arora; and Oligonucleotide-Based Drugs and Therapeutics: Preclinical and Clinical Considerations by N. Ferrari and R. Segui.

Inhibitory nucleic acids can also include RNA aptamers, which are short, synthetic oligonucleotide sequences that bind to proteins (see. e.g., Li et al., Nuc. Acids Res. (2006), 34:6416-24). They are notable for both high affinity and specificity for the targeted molecule, and have the additional advantage of being smaller than antibodies (usually less than 6 kD). RNA aptamers with a desired specificity are generally selected from a combinatorial library, and can be modified to reduce vulnerability to ribonucleases, using methods known in the art.

### Antibodies

In some embodiments, the agent is an anti-15-PGDH antibody or an antigen-binding fragment thereof. In some embodiments, the antibody is a blocking antibody (e.g., an antibody that binds to a target and directly interferes with the target's function, e.g., 15-PGDH enzyme activity). In some embodiments, the antibody is a neutralizing antibody (e.g., an antibody that binds to a target and negates the downstream cellular effects of the target). In some embodiments, the antibody binds to human 15-PGDH.

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)₂, Fab′, Fab, scFv, and the like. The term “antibody or antigen-binding fragment” can also encompass multi-specific and hybrid antibodies, with dual or multiple antigen or epitope specificities.

In some embodiments, an anti-15-PGDH antibody comprises a heavy chain sequence or a portion thereof, and/or a light chain sequence or a portion thereof, of an antibody sequence disclosed herein. In some embodiments, an anti-15-PGDH antibody comprises one or more complementarity determining regions (CDRs) of an anti-15-PGDH antibody as disclosed herein. In some embodiments, an anti-15-PGDH antibody is a nanobody, or single-domain antibody (sdAb), comprising a single monomeric variable antibody domain, e.g., a single VHH domain.

For preparing an antibody that binds to 15-PGDH, many techniques known in the art can be used. See. e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies. A Laboratory Manual (1988); and Goding, Monoclonal Antibodies: Principles and Practice (2nd ed. 1986)). In some embodiments, antibodies are prepared by immunizing an animal or animals (such as mice, rabbits, or rats) with an antigen for the induction of an antibody response. In some embodiments, the antigen is administered in conjugation with an adjuvant (e.g., Freund's adjuvant). In some embodiments, after the initial immunization, one or more subsequent booster injections of the antigen can be administered to improve antibody production. Following immunization, antigen-specific B cells are harvested, e.g., from the spleen and/or lymphoid tissue. For generating monoclonal antibodies, the B cells are fused with myeloma cells, which are subsequently screened for antigen specificity.

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Additionally, phage or yeast display technology can be used to identify antibodies and heteromeric Fab fragments that specifically bind to selected antigens (see. e.g., McCafferty et al., Nature 348:552-554 (1990); Marks et al., Biotechnology 10:779-783 (1992); Lou et al., PEDS 23:311 (2010); and Chao et al., Nature Protocols 1:755-768 (2006)). Alternatively, antibodies and antibody sequences may be isolated and/or identified using a yeast-based antibody presentation system, such as that disclosed in, e.g., Xu et al., Protein Eng Des Sel, 2013, 26:663-670; WO 2009/036379: WO 2010/105256; and WO 2012/009568. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can also be adapted to produce antibodies.

Antibodies can be produced using any number of expression systems, including prokaryotic and eukaryotic expression systems. In some embodiments, the expression system is a mammalian cell, such as a hybridoma, or a CHO cell. Many such systems are widely available from commercial suppliers. In embodiments in which an antibody comprises both a VH and VL region, the VH and VL regions may be expressed using a single vector, e.g., in a di-cistronic expression unit, or be under the control of different promoters. In other embodiments, the VH and VL region may be expressed using separate vectors.

In some embodiments, an anti-15-PGDH antibody comprises one or more CDR, heavy chain, and/or light chain sequences that are affinity matured. For chimeric antibodies, methods of making chimeric antibodies are known in the art. For example, chimeric antibodies can be made in which the antigen binding region (heavy chain variable region and light chain variable region) from one species, such as a mouse, is fused to the effector region (constant domain) of another species, such as a human. As another example, “class switched” chimeric antibodies can be made in which the effector region of an antibody is substituted with an effector region of a different immunoglobulin class or subclass.

In some embodiments, an anti-15-PGDH antibody comprises one or more CDR, heavy chain, and/or light chain sequences that are humanized. For humanized antibodies, methods of making humanized antibodies are known in the art. See, e.g., U.S. Pat. No. 8,095,890. Generally, a humanized antibody has one or more amino acid residues introduced into it from a source which is non-human. As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)₂, a scFv, nanobody, or a diabody) are generated. Various techniques have been developed for the production of antibody fragments, such as proteolytic digestion of intact antibodies (see, e.g., Morimoto et al., J. Biochem. Biophys. Meth., 24:107-117 (1992); and Brennan et al., Science, 229:81 (1985)) and the use of recombinant host cells to produce the fragments. For example, antibody fragments can be isolated from antibody phage libraries. Alternatively, Fab′-SH fragments can be directly recovered from E. coli cells and chemically coupled to form F(ab′)₂ fragments (see. e.g., Carter el al., BioTechnology, 10:163-167 (1992)). According to another approach, F(ab′)₂ fragments can be isolated directly from recombinant host cell culture. Other techniques for the production of antibody fragments will be apparent to those skilled in the art.

Methods for measuring binding affinity and binding kinetics are known in the art. These methods include, but are not limited to, solid-phase binding assays (e.g., ELISA assay), immunoprecipitation, surface plasmon resonance (e.g., Biacore™ (GE Healthcare, Piscataway, NJ)), kinetic exclusion assays (e.g., KinExA®), flow cytometry, fluorescence-activated cell sorting (FACS), BioLayer interferometry (e.g., Octet™ (ForteBio, Inc., Menlo Park, CA)), and western blot analysis.

### Peptides

In some embodiments, the agent is a peptide, e.g., a peptide that binds to and/or inhibits the enzymatic activity or stability of 15-PGDH. In some embodiments, the agent is a peptide aptamer. Peptide aptamers are artificial proteins that are selected or engineered to bind to specific target molecules. Typically, the peptides include one or more peptide loops of variable sequence displayed by the protein scaffold. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. See, e.g., Reverdatto et al., 2015, Curr. Top. Med. Chem. 15:1082-1101.

In some embodiments, the agent is an affimer. Affimers are small, highly stable proteins, typically having a molecular weight of about 12-14 kDa, that bind their target molecules with specificity and affinity similar to that of antibodies. Generally, an affimer displays two peptide loops and an N-terminal sequence that can be randomized to bind different target proteins with high affinity and specificity in a similar manner to monoclonal antibodies. Stabilization of the two peptide loops by the protein scaffold constrains the possible conformations that the peptides can take, which increases the binding affinity and specificity compared to libraries of free peptides. Affimers and methods of making affimers are described in the art. See, e.g., Tiede et al., eLife, 2017, 6:e24903. Affimers are also commercially available, e.g., from Avacta Life Sciences.

### Vectors and Modified RNA

In some embodiments, polynucleotides providing 15-PGDH inhibiting activity, e.g., a nucleic acid inhibitor such as an siRNA or shRNA, or a polynucleotide encoding a polypeptide that inhibits 15-PGDH, are introduced into cells, e.g., tissue cells, using an appropriate vector. Examples of delivery vectors that may be used with the present disclosure are viral vectors, plasmids, exosomes, liposomes, bacterial vectors, or nanoparticles. In some embodiments, any of the herein-described 15-PGDH inhibitors, e.g., a nucleic acid inhibitor or a polynucleotide encoding a polypeptide inhibitor, are introduced into cells, e.g., tissue cells, using vectors such as viral vectors. Suitable viral vectors include but not limited to adeno-associated viruses (AAVs), adenoviruses, and lentiviruses. In some embodiments, a 15-PGDH inhibitor, e.g., a nucleic acid inhibitor or a polynucleotide encoding a polypeptide inhibitor, is provided in the form of an expression cassette, typically recombinantly produced, having a promoter operably linked to the polynucleotide sequence encoding the inhibitor. In some cases, the promoter is a universal promoter that directs gene expression in all or most tissue types; in other cases, the promoter is one that directs gene expression specifically in cells of the tissue being targeted.

In some embodiments, the nucleic acid or protein inhibitors of 15-PGDH are introduced into a subject, e.g., into the tissues of a subject, using modified RNA. Various modifications of RNA are known in the art to enhance, e.g., the translation, potency and/or stability of RNA, e.g., shRNA or mRNA encoding a 15-PGDH polypeptide inhibitor, when introduced into cells of a subject. In particular embodiments, modified mRNA (mmRNA) is used, e.g., mmRNA encoding a polypeptide inhibitor of 15-PGDH. In other embodiments, modified RNA comprising an RNA inhibitor of 15-PGDH expression is used, e.g., siRNA, shRNA, or miRNA. Non-limiting examples of RNA modifications that can be used include anti-reverse-cap analogs (ARCA), polyA tails of, e.g., 100-250 nucleotides in length, replacement of AU-rich sequences in the 3′UTR with sequences from known stable mRNAs. and the inclusion of modified nucleosides and structures such as pseudouridine, e.g., N1-methylpseudouridine, 2-thiouridine, 4′thioRNA, 5-methylcytidine, 6-methyladenosine, amide 3 linkages, thioate linkages, inosine, 2′-deoxyribonucleotides, 5-Bromo-uridine and 2′-O-methylated nucleosides. A non-limiting list of chemical modifications that can be used can be found, e.g., in the online database crdd.osdd.net/servers/sirnamod/. RNAs can be introduced into cells in vivo using any known method, including, inter alia, physical disturbance, the generation of RNA endocytosis by cationic carriers, electroporation, gene guns, ultrasound, nanoparticles, conjugates, or high-pressure injection. Modified RNA can also be introduced by direct injection, e.g., in citrate-buffered saline. RNA can also be delivered using self-assembled lipoplexes or polyplexes that are spontaneously generated by charge-to-charge interactions between negatively charged RNA and cationic lipids or polymers, such as lipoplexes, polyplexes, polycations and dendrimers. Polymers such as poly-L-lysine, polyamidoamine, and polyethyleneimine, chitosan, and poly(3-amino esters) can also be used. See. e.g., Youn et al. (2015) Expert Opin Biol Ther, September 2; 15(9): 1337-1348; Kaczmarek et al. (2017) Genome Medicine 9:60; Gan et al. (2019) Nature comm. 10: 871; Chien et al. (2015) Cold Spring Harb Perspect Med. 2015; 5:a014035; the entire disclosures of each of which are herein incorporated by reference.

### 8. Methods of Administration

The compounds described herein can be administered locally in the subject or systemically. In some embodiments, the compounds can be administered, for example, intraperitoneally, intramuscularly, intra-arterially, orally, intravenously, intracranially, intrathecally, intraspinally, intralesionally, intranasally, subcutaneously, intracerebroventricularly, topically, and/or by inhalation. In an example, the compounds are administered intramuscularly, e.g., by intramuscular injection.

In some embodiments, the compound is administered in accordance with an acute regimen. In certain instances, the compound is administered to the subject once. In other instances, the compound is administered at one time point, and administered again at a second time point. In yet other instances, the compound is administered to the subject repeatedly (e.g., once or twice daily) as intermittent doses over a short period of time (e.g., 2 days, 3 days, 4 days, 5 days, 6 days, a week, 2 weeks, 3 weeks, 4 weeks, a month, or more). In some cases, the time between compound administrations is about 1 day, 2 days, 3 days, 4 days, 5 days, 6 days, a week, 2 weeks, 3 weeks, 4 weeks, a month, or more. In other embodiments, the compound is administered continuously or chronically in accordance with a chronic regimen over a desired period of time. For instance, the compound can be administered such that the amount or level of the compound is substantially constant over a selected time period.

Administration of the compound into a subject can be accomplished by methods generally used in the art. The quantity of the compound introduced may take into consideration factors such as sex, age, weight, the types of disease or disorder, stage of the disorder, and the quantity needed to produce the desired result. Generally, for administering the compound for therapeutic purposes, the cells are given at a pharmacologically effective dose. By “pharmacologically effective amount” or “pharmacologically effective dose” is an amount sufficient to produce the desired physiological effect or amount capable of achieving the desired result, particularly for treating the condition or disease, including reducing or eliminating one or more symptoms or manifestations of the condition or disease.

The compounds described herein may be administered locally by injection into the tissue being targeted, or by administration in proximity to the tissue being targeted.

### 9. Pharmaceutical Compositions

The pharmaceutical compositions of the compounds described herein may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions described herein (see, e.g., REMINGTON'S PHARMACEUTICAL SCIENCES, 18TH ED., Mack Publishing Co., Easton, Pa. (1990)).

As used herein, “pharmaceutically acceptable carrier” comprises any of standard pharmaceutically accepted carriers known to those of ordinary skill in the art in formulating pharmaceutical compositions. Thus, the compounds, by themselves, such as being present as pharmaceutically acceptable salts, or as conjugates, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

The pharmaceutical compositions will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions described herein are administered in a manner compatible with the dosage formulation, and in such amount as will be therapeutically effective. The quantity to be administered depends on a variety of factors including, e.g., the age, body weight, physical activity, and diet of the individual, the condition or disease to be treated, and the stage or severity of the condition or disease. In certain embodiments, the size of the dose may also be determined by the existence, nature, and extent of any adverse side effects that accompany the administration of a therapeutic agent(s) in a particular individual.

It should be understood, however, that the specific dose level and frequency of dosage for any particular patient may be varied and may depend upon a variety of factors including the activity of the specific compound employed, the metabolic stability and length of action of that compound, the age, body weight, hereditary characteristics, general health, sex, diet, mode and time of administration, rate of excretion, drug combination, the severity of the particular condition, and the host undergoing therapy.

In certain embodiments, the dose of the compound may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least about 1, about 2, about 3, about 4, about 5, about 6, about 7, about 8, about 9, about 10, or more times the amount of the therapeutic compound.

Methods for preparing such dosage forms are known to those skilled in the art (see, e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (e.g., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

In some embodiments, additional compounds or medications can be co-administered to the subject. Such compounds or medications can be co-administered for the purpose of alleviating signs or symptoms of the disease being treated, reducing side-effects caused by induction of the immune response, etc. In some embodiments, for example, the 15-PGDH inhibitors described herein are administered together with a compound to enhance PGE2 levels and/or PGD2 levels, a compound to increase signaling through the EP1, EP2, EP3, EP4, DP1, and/or DP2 receptors, and/or any other compound aiming to enhance mitochondrial biogenesis and function or the function, health, or any other desired property of the tissue being targeted.

### 10. Kits

Other embodiments of the compositions described herein are kits comprising a 15-PGDH inhibitor. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kit comprises one or more reagents for increasing mitochondrial biogenesis and function in a tissue of a subject. In some embodiments, the kit comprises one or more reagents for the treatment of a disease associated with mitochondrial dysfunction and/or oxidative stress such as a mitochondrial disease. In some embodiments, the kit comprises an agent that antagonizes the expression or activity of 15-PGDH. In some embodiments, the kit comprises an inhibitory nucleic acid (e.g., an antisense RNA, small interfering RNA (siRNA), microRNA (miRNA), short hairpin RNA (shRNA)), or a polynucleotide encoding a 15-PGDH inhibiting polypeptide, that inhibits or suppresses 15-PGDH mRNA or protein expression or activity, e.g., enzyme activity. In some embodiments, the kit comprises a modified RNA, e.g., a modified shRNA or siRNA, or a modified mRNA encoding a polypeptide 15-PGDH inhibitor. In some embodiments, the kit further comprises one or more plasmid, bacterial or viral vectors for expression of the inhibitory nucleic acid or polynucleotide encoding a 15-PGDH-inhibiting polypeptide. In some embodiments, the kit comprises an antisense oligonucleotide capable of hybridizing to a portion of a 15-PGDH-encoding mRNA. In some embodiments, the kit comprises an antibody (e.g., a monoclonal, polyclonal, humanized, bispecific, chimeric, blocking or neutralizing antibody) or antibody-binding fragment thereof that specifically binds to and inhibits a 15-PGDH protein. In some embodiments, the kit comprises a blocking peptide. In some embodiments, the kit comprises an aptamer (e.g., a peptide or nucleic acid aptamer). In some embodiments, the kit comprises an affimer. In some embodiments, the kit comprises a modified RNA. In particular embodiments, the kit comprises a small molecule inhibitor, e.g., SW033291, that binds to 15-PGDH or inhibits its enzymatic activity. In some embodiments, the kit further comprises one or more additional therapeutic agents, e.g., agents for administering in combination therapy with the agent that antagonizes the expression or activity of 15-PGDH.

In some embodiments, the kits can further comprise instructional materials containing directions (e.g., protocols) for the practice of the methods described herein (e.g., instructions for using the kit for increasing mitochondrial biogenesis and function in a tissue of a subject; and/or for using the kit for the treatment of a disease associated with mitochondrial dysfunction and/or oxidative stress such as a mitochondrial disease). While the instructional materials typically comprise written or printed materials, they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The present disclosure will be described in greater detail by way of a specific example. The following example is offered for illustrative purposes only, and is not intended to limit the disclosure in any manner. Those of skill in the art will readily recognize a variety of noncritical parameters which can be changed or modified to yield essentially the same results.

### Example 1. Elevation of Mitochondrial Function and Biogenesis and Increase in Autophagy Following 15-PGDH Inhibition

### Results

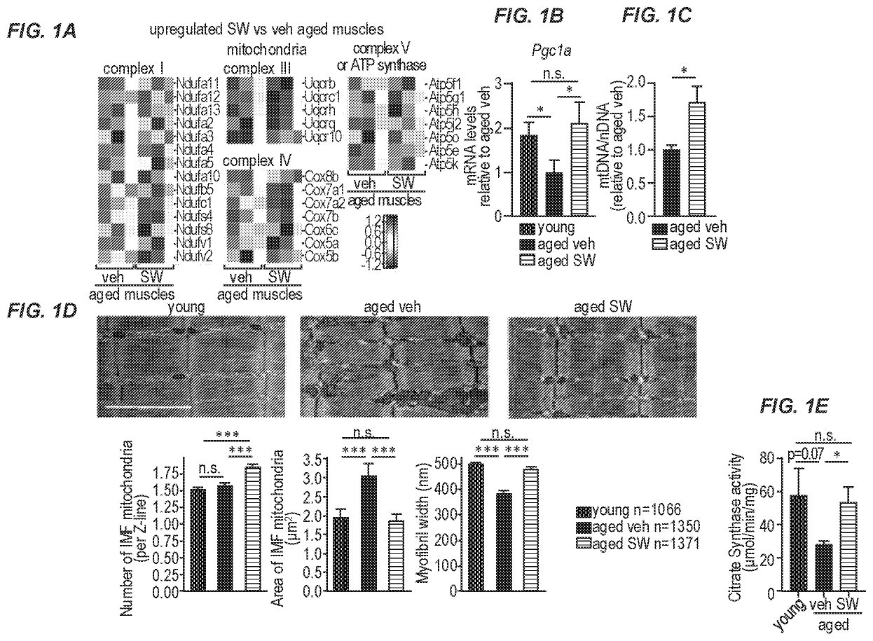

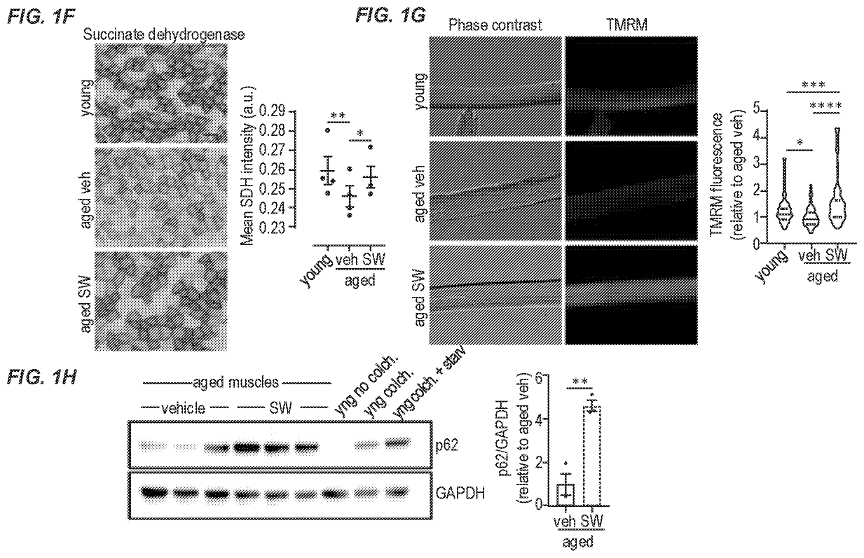

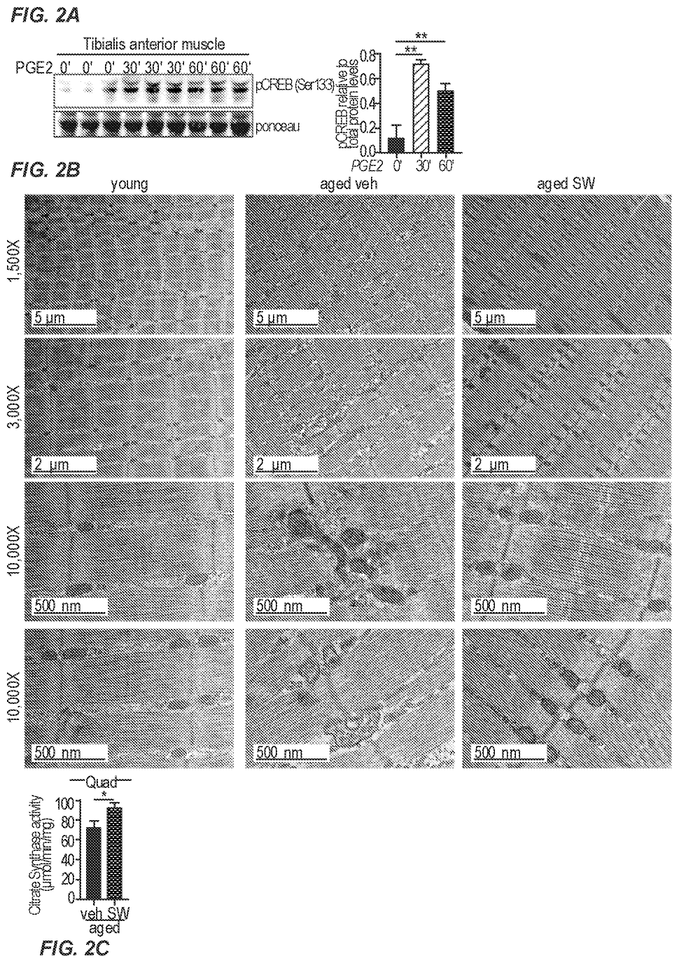

To identify downstream signaling pathways through which 15-PGDH inhibition exerts its effects on aged muscles, we performed an unbiased transcriptomic analysis of aged muscles treated with vehicle or SW033291 (SW). Among the most striking changes in the aged muscle tissue transcriptome after SW treatment was a strong enrichment for mitochondrial pathways, including mitochondrial oxidative phosphorylation, ATP synthesis and other metabolic and energy producing processes (FIG. 1A). Numerous components of the mitochondria complexes I, II, IV and V of the electron transport chain were markedly increased in SW-treated aged muscles (FIG. 1A). PGE2 signaling through the G-coupled protein receptor, EP4, is known to be mediated by cyclic AMP (cAMP) (D. Wang, R. N. Dubois, Eicosanoids and cancer. Nat Rev Cancer 10, 181-193 (2010); M. Korotkova, I. E. Lundberg, The skeletal muscle arachidonic acid cascade in health and inflammatory disease. Nat Rev Rheumatol 10, 295-303 (2014); E. Ricciotti, G. A. FitzGerald, Prostaglandins and inflammation. Arterioscler Thromb Vasc Biol 31, 986-1000 (2011)). We confirmed that PGE2 activates the cyclic AMP response element binding protein (CREB) in skeletal muscles (FIG. 2A). Importantly, when we assayed mRNA levels of the master regulator for mitochondrial biogenesis that has a CREB binding motif in its promoter, peroxisome proliferator-activated receptor gamma coactivator 1-alpha (Pgc1α) (P. J. Fernandez-Marcos, J. Auwerx, Regulation of PGC-1alpha, a nodal regulator of mitochondrial biogenesis. Am J Clin Nutr 93, 884S-890 (2011)), we found that its level was restored to that seen in young muscles (FIG. 1B). Overall mitochondrial content was increased, as reflected by the increased mitochondrial DNA content relative to nuclear DNA content following SW treatment of aged muscles (FIG. 1C).

In accordance, a morphological assessment by transmission electron microscopy (TEM) of Extensor digitorum longus (EDL) muscles after SW treatment revealed an increase in the number of intermyofibrillar (IMF) mitochondria (FIGS. 1D and 2B). In addition, the morphology of the mitochondria improved. The shift in IMF mitochondrial area from compact and round in young to a larger size with more disorganized morphology in aged tissues was clearly evident, in agreement with reports by others (J. P. Leduc-Gaudet et al., Mitochondrial morphology is altered in atrophied skeletal muscle of aged mice. Oncotarget 6, 17923-17937 (2015); V. Romanello, M. Sandri, Mitochondrial Quality Control and Muscle Mass Maintenance. Front Physiol 6, 422 (2015)) (FIGS. 1D and 2B). Following one month of SW treatment mitochondrial morphology was restored to a compact mitochondrial morphology resembling that seen in young (FIG. 1D). TEM analysis also revealed an increase in myofibril width consistent with the increased cross sectional area and muscle mass and overall attenuation of muscle atrophy seen in the aged treated with SW compared to vehicle controls (FIGS. 1D and 2B).

To assess mitochondrial function, we analyzed citrate synthase activity, the first enzyme of the Krebs cycle (J. M. Berg. J. L. Tymoczko, L. Stryer, Biochemistry. (W.H. Freeman, 2002)), in isolated mitochondria and found that it was significantly increased in SW-treated aged muscles compared to controls, reaching levels comparable to young (FIGS. 1E and 2C). SW treated aged muscle mitochondria also expressed increased succinate dehydrogenase activity, a key component of both the Krebs cycle and the electron transport chain (J. M. Berg, J. L. Tymoczko, L. Stryer, Biochemistry. (W.H. Freeman, 2002)) (FIG. 1F). Additionally, we observed an increase in mitochondrial membrane potential in myofibers isolated from EDL muscles of SW treated aged mice compared to controls (FIG. 1G). Together, these data provide strong evidence that a one month 15-PGDH inhibition and consequent elevation of PGE2 in aged muscles triggers mitochondrial biogenesis and increased mitochondrial function to meet the energetic requirements of muscle growth. A decrease in autophagy and an accumulation of dysfunctional organelles such as mitochondria has been associated with sarcopenia (V. Romanello, M. Sandri, Mitochondrial Quality Control and Muscle Mass Maintenance. Front Physiol 6, 422 (2015)). In accordance with the increase in mitochondrial function, we observed an increase in autophagy flux. Evidence for autophagy was shown by a standard assay, western blot of increased p62 upon colchicine treatment of muscles of aged mice treated with SW compared to controls (FIG. 1H). Taken together these data indicate that SW treatment and the consequent systemic elevation of endogenous PGE2 levels enhances the energetic capacity of skeletal muscles.

### Discussion

Our transcriptome analysis comparing aged muscles following a one-month treatment with a small molecule inhibitor of 15-PGDH with vehicle treated controls revealed that mitochondrial function is among the top upregulated pathways. PGE2 signaling through the EP4 receptor via cAMP/CREB could account for the observed increase in mitochondrial number and function, in agreement with prior reports (D. Wang, R. N. Dubois, Eicosanoids and cancer. Nat Rev Cancer 10, 181-193 (2010); M. Korotkova, I. E. Lundberg, The skeletal muscle arachidonic acid cascade in health and inflammatory disease. Nat Rev Rheumatol 10, 295-303 (2014); E. Ricciotti, G. A. FitzGerald. Prostaglandins and inflammation. Arterioscler Thromb Vasc Biol 31, 986-1000 (2011)). Similar to the beneficial effects on skeletal muscle previously shown for other cAMP inducing agents, such as β-adrenergic receptor (β-AR) agonists or corticotropin releasing factor receptor 2 (CRFR2) agonists, PGE2 induction of cAMP likely augments mitochondrial function by activating downstream transcriptional regulators with cAMP response elements (CREB binding motifs) that promote mitochondrial biogenesis, including the master mitochondrial regulator Pgc1α and other oxidative genes (R. Berdeaux, R. Stewart, cAMP signaling in skeletal muscle adaptation: hypertrophy, metabolism, and regeneration. Am J Physiol Endocrinol Metab 303, E1-17 (2012); S. Herzig et al., CREB regulates hepatic gluconeogenesis through the coactivator PGC-1. Nature 413, 179-183 (2001); S. Austin, J. St-Pierre, PGC1alpha and mitochondrial metabolism—emerging concepts and relevance in ageing and neurodegenerative disorders. J Cell Sci 125, 4963-4971 (2012)). In addition to increased mitochondrial gene expression, the increase in mitochondrial function upon SW treatment is striking and likely plays a major role in countering muscle atrophy with aging. All mitochondrial functions assayed were decreased in aged relative to young muscles and restored after one month of SW treatment. The number of mitochondria was increased and had a compact morphology in stark contrast with the significantly increased size and distended aberrant morphology of aged muscle mitochondria. The decreased activity of key Krebs cycle mitochondrial enzymes, citrate synthase and succinate dehydrogenase, and mitochondrial membrane potential in aged, were restored to young levels by SW treatment. Thus, 15-PGDH inhibition triggers a signaling cascade that culminates in a dramatic effect on mitochondrial biogenesis and improved mitochondrial function together with a marked reversal of muscle atrophy.

### Example 2. Rejuvenating Brown Fat and Triggering Mitochondrial Biogenesis by Administration of a 15-PGDH Inhibitor

In this example, an individual presents at a healthcare facility with decreased brown fat function. The individual is a 30-year-old with obesity. The individual is treated with a therapeutically effective amount of a 15-PGDH inhibitor (e.g., in an amount effective to reduce 15-PGDH levels in the individual or an amount effective to inhibit 15-PGDH activity in the individual). The individual is treated by systemic administration (e.g., orally) of the 15-PGDH inhibitor, or by local administration (e.g., directly to the brown fat, e.g., by injection) of the 15-PGDH inhibitor. After administration of the 15-PGDH inhibitor, the individual exhibits rejuvenation of one or more functions or properties of the aged brown fat, e.g., relative to prior to the administration (e.g., the function is rejuvenated to a level substantially similar to a level found in young brown fat), e.g., as measured by medical imaging, e.g., MRI or fMRI. The individual exhibits increased levels of UCP1 (e.g., as determined by assaying a sample collected from the individual). The individual exhibits brown fat with increased mitochondrial activity and/or concentration (e.g., as determined by microscopy of a biopsy sample taken from the individual). The 15-PGDH inhibitor triggers mitochondrial biogenesis and increased mitochondrial function.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of increasing mitochondrial biogenesis, mitochondrial function, or both in a tissue of a subject, the method comprising: administering to the subject an amount of a 15-hydroxyprostaglandin dehydrogenase (15-PGDH) inhibitor effective to inhibit 15-PGDH activity and/or reduce 15-PGDH levels in the subject, thereby increasing mitochondrial biogenesis, mitochondrial function, or both in the tissue of the subject.

2. The method of claim 1, wherein the method comprises increasing mitochondrial biogenesis.

3. The method of claim 1, wherein the method comprises increasing mitochondrial function.

4. The method of any one of claims 1-3, wherein the administering comprises modulating one or more energy biomarkers, normalizing one or more energy biomarkers, enhancing one or more energy biomarkers, or any combination thereof.

5. The method of claim 3, wherein the one or more energy biomarkers are selected from the group consisting of: whole blood, plasma, cerebrospinal fluid, and/or cerebral ventricular fluid lactic acid (lactate) levels; whole blood, plasma, cerebrospinal fluid, and/or cerebral ventricular fluid pyruvic acid (pyruvate) levels; whole blood, plasma, cerebrospinal fluid, and/or cerebral ventricular fluid lactate/pyruvate ratios; phosphocreatine levels; NADH (NADH+H+) levels; NADPH (NADPH+H+) levels; NAD levels; NADP levels; ATP levels; reduced coenzyme Q (CoQred) levels; oxidized coenzyme Q (CoQox) levels; total coenzyme Q (CoQlot) levels, oxidized cytochrome C levels; reduced cytochrome C levels; oxidized cytochrome C/reduced cytochrome C ratio; acetoacetate levels; beta-hydroxy butyrate levels; acetoacetate/beta-hydroxy butyrate ratio; 8-hydroxy-2′-deoxyguanosine (8-OHdG) levels; levels of reactive oxygen species; levels of oxygen consumption (VO2); levels of carbon dioxide output (VCO2); respiratory quotient (VCO2/VO2); exercise tolerance; and anaerobic threshold.

6. The method of any one of claims 1-5, wherein the administering increases a level of prostaglandin E₂ (PGE2) in the tissue of the subject.

7. The method of claim 6, wherein the level of PGE2 in the tissue is increased relative to the tissue prior to the administering of the 15-PGDH inhibitor.

8. The method of any one of claims 1-7, wherein the subject has a mitochondrial disease, disorder, or condition.

9. The method of claim 8, wherein the mitochondrial disease, disorder, or condition is caused by mitochondrial dysfunction and/or oxidative stress.

10. The method of claim 9, wherein the administering reduces mitochondrial dysfunction and/or oxidative stress in the tissue of the subject.

11. The method of claim 8, wherein the mitochondrial disease, disorder, or condition is selected from the group consisting of a metabolic disease, a neurodegenerative disease, cancer, a motor neuron disease, a cardiovascular disease, a neurobehavioral or psychiatric disease, an autoimmune disease, a chronic kidney disease, myoclonic epilepsy with ragged red fibers (MERRF), mitochondrial myopathy, dominant optic atrophy (DOA), Leigh syndrome, Kearns-Sayre Syndrome (KSS), Friedrich's ataxia (FRDA), cardiomyopathy, renal tubular acidosis, Parkinson's disease, Alzheimer's disease, amyotrophic lateral sclerosis (ALS), Huntington's disease, developmental pervasive disorders, hearing loss, mitochondrial encephalopathy lactic acidosis and stroke-like episodes (MELAS), chronic obstructive pulmonary disease (COPD), ventilator induced diaphragmatic dysfunction (VIDD), myalgic encephalomyelitis/chronic fatigue syndrome, Leber hereditary optic neuropathy (LHON), chemotherapy-induced peripheral neuropathy, and a combination thereof.

12. The method of any one of claims 7-11, wherein the level of PGE2 in the tissue is increased to a level substantially similar to a level present in the same tissue of a subject not having the mitochondrial disease, disorder, or condition.

13. The method of any one of claims 7-12, wherein the level of PGE2 in the tissue is increased to a level within 50% of a level present in the same tissue of a subject not having the mitochondrial disease, disorder, or condition.

14. The method of any one of claims 1-13, wherein the tissue is selected from the group consisting of skeletal muscle, epidermal tissue, epithelial tissue, vascular tissue, cardiac muscle, brain, bone, cartilage, sensory organs, kidney, thyroid, lung, smooth muscle, brown fat, spleen, liver, heart, small intestine, colon, skin, ovaries and other reproductive tissues, hair, dental tissue, blood, cochlea, and a combination thereof.

15. The method of any one of claims 1-14, wherein mitochondrial biogenesis, function, or both in the tissue is increased relative to the tissue prior to the administering of the 15-PGDH inhibitor.

16. The method of any one of claims 1-15, wherein the method results in an increased level of peroxisome proliferator-activated receptor gamma coactivator 1-alpha (Pgc1α) in the tissue, increased mitochondrial DNA relative to nuclear DNA in the tissue, increased number of mitochondria in the tissue, improved mitochondrial morphology in the tissue, increased mitochondrial complexes, enzymes involved in ATP generation, electron transport, and/or respiration in mitochondria present in the tissue, increased activity of complexes I, II, III, IV, and/or V of the electron transport chain in mitochondria present in the tissue, increased mitochondrial membrane potential in the tissue, or any combination thereof.

17. The method of any one of claims 1-16, wherein the method results in increased mitophagy or autophagy leading to mitochondria turnover in the tissue.

18. The method of any one of claims 1-17, wherein the method results in a decreased level of a PGE2 metabolite in the tissue relative to the tissue prior to the administering of the 15-PGDH inhibitor.

19. The method of any one of claims 7-18, wherein the method results in a level of a PGE2 metabolite in the tissue that is substantially similar to a level present in the same tissue of a subject not having the mitochondrial disease.

20. The method of claim 18 or 19, wherein the PGE2 metabolite is selected from the group consisting of 15-keto PGE2 and 13,14-dihydro-15-keto PGE2.

21. The method of any one of claims 1-20, wherein the 15-PGDH inhibitor is selected from the group consisting of a small molecule compound, a blocking antibody, a nanobody, and a peptide.

22. The method of any one of claims 1-21, wherein the 15-PGDH inhibitor is SW033291.

23. The method of any one of claims 1-21, wherein the 15-PGDH inhibitor is selected from the group consisting of an antisense oligonucleotide, microRNA, siRNA, and shRNA.

24. The method of any one of claims 1-23, wherein the subject is a human.

25. The method of any one of claims 1-24, wherein the subject is less than 30 years of age.

26. The method of any one of claims 1-24, wherein the subject is at least 30 years of age.

27. The method of any one of claims 1-26, wherein the 15-PGDH inhibitor reduces or blocks 15-PGDH expression.

28. The method of any one of claims 1-27, wherein the 15-PGDH inhibitor reduces or blocks enzymatic activity of 15-PGDH.

29. A method of improving a metabolic function in a tissue of a subject having an abnormal metabolism, the method comprising administering to the subject an amount of a 15-PGDH inhibitor effective to inhibit 15-PGDH activity and/or reduce 15-PGDH levels in the subject, thereby improving the metabolic function in the tissue of the subject having the abnormal metabolism.

30. The method of claim 29, wherein the 15-PGDH inhibitor reduces or blocks enzymatic activity of 15-PGDH.

31. The method of claim 29 or 30, wherein the abnormal metabolism comprises a metabolic disorder or an obesity-related disorder.

32. The method of claim 31, wherein the metabolic disorder is Metabolic syndrome (MS).

33. The method of claim 31, wherein the metabolic disorder or the obesity-related disorder comprises a disorder that results in or causes obesity.

34. The method of claim 33, wherein the disorder that results in or causes obesity is selected from the group consisting of overeating and bulimia, polycystic ovarian disease, craniopharyngioma, Prader-Willi Syndrome, Frohlich's syndrome, Type II diabetics, GH-deficient subjects, normal variant short stature, Turner's syndrome, acute lymphoblastic leukemia, and other pathological conditions showing reduced metabolic activity or a decrease in resting energy expenditure as a percentage of total fat-free mass.

35. The method of claim 31, wherein the metabolic disorder or obesity-related disorder is selected from the group consisting of insulin resistant obesity, diabetes, hyperphagia, endocrine abnormalities, triglyceride storage disease, Bardet-Biedl syndrome, Lawrence-Moon syndrome, Prader-Labhard-Willi syndrome, anorexia, and cachexia.

36. The method of claim 29 or 30, wherein the subject experiences aberrant thermogenesis or aberrant adipose cell content or function.

37. The method of claim 29, wherein the abnormal metabolism comprises misregulation of Pgc1α activity.

38. The method of claim 37, wherein the misregulation of Pgc1α activity comprises downregulation of Pgc1α activity.

39. The method of claim 37, wherein the misregulation of Pgc1α activity comprises upregulation of Pgc1α activity.

40. The method of any one of claims 29-39, wherein the improving the metabolic function comprises increasing mitochondrial biogenesis.

41. The method of any one of claims 29-39, wherein the metabolic function comprises the functioning of mitochondria.

42. The method of claim 29, wherein the improving the metabolic function comprises increasing mitophagy.

43. The method of claim 29, wherein the improving the metabolic function comprises enriching mitochondrial oxidative phosphorylation or ATP synthesis.

44. The method of any one of claims 29-43, wherein the metabolic function is selected from the group consisting of cellular proliferation, growth, differentiation, or migration, cellular regulation of homeostasis; inter- or intra-cellular communication; tissue function; and systemic response in an organism.

45. The method of claim 44, wherein the tissue function comprises liver function, muscle function, or adipocyte function.

46. The method of claim 44, wherein the systemic response in an organism comprises a hormonal response.

47. The method of claim 46, wherein the hormonal response comprises an insulin response.

48. The method of claim 29, wherein the abnormal metabolism comprises a condition selected from the group consisting of inherited mitochondrial diseases, MERRF, MELAS, LHON, DOA, Leigh syndrome, Kearns-Sayre syndrome, Friedreich's ataxia, other myopathies, cardiomyopathy, encephalomyopathy, renal tubular acidosis, neurodegenerative diseases, Parkinson's disease, Alzheimer's disease, ALS, motor neuron diseases, epilepsy, genetic diseases, Huntington's Disease, mood disorders, schizophrenia, bipolar disorder, and age-associated diseases.
